(19) United States
(12) Patent Application Publication
(10) Pub. No.: US 2006/0172300 A1
(43) Pub. Date: Aug. 3, 2006

(54) Method for identifying bhs-specific proteins and fragments thereof

(75) Inventors: Sabine Wolf, Otzberg/Lengfeld (DE); Martina Jager, Muhtal (DE); Thorsten Bangsow, Buttelborn-Worfelden (DE); Carmen Bangsow, Buttelborn-Worfelden (DE); Dominik Jordan, Pflaumheim (DE); Bernhard Pelzer, Darmstadt (DE); Thomas Oppolzer, Darmstadt (DE)

Correspondence Address: EDWARDS & ANGELL, LLP, P.O. BOX 55874, BOSTON, MA, 02205, US

(73) Assignee: FrankGen Biotechnologie AG, Kronberg (DE)

(21) Appl. No.: 10/527,788

(22) PCT Filed: Mar. 8, 2003
(86) PCT No.: PCT/EP03/09968

§ 371 Date: Nov. 2, 2005

(30) Foreign Application Priority Data
Sep. 11, 2002 (DE) 102 42 016.5

(51) Int. Cl. C12Q 1/68; G01N 33/00; C07K 14/705

(52) U.S. Cl. 435/6; 436/86; 530/350

(57) ABSTRACT

The invention relates to a method for identifying the presence of BBB-specific protein/fragment in endothelial cells of brain capillaries, characterized in that a) endothelial cells of brain capillaries freshly isolated from brain are conventionally pre-purified by enzymatic digestion, b) the digest obtained in step a) is treated with a lysis buffer that essentially destroys present erythrocytes and apoptotic cells and maintains at least 70% of the endothelial cells of brain capillaries in vital form, c) the product obtained in step b) is optionally purified further, d) a subtractive cDNA library is prepared from the endothelial cells of brain capillaries and a subtractive tissue, e) a cDNA subtraction is performed using one ore more differential hybridization(s), f) clones from the subtractive cDNA library are verified by differential hybridization with respect to their respective expression, g) a complete cDNA sequence is prepared for the BBB-specific clones from the subtractive cDNA library, and h) the expression pattern of the investigated clones is compared between fresh and cultured endothelial cells of brain capillaries and, that way, the presence of BBB-specific proteins or fragments thereof is identified as well as proteins and fragments thereof identified with this method.

The invention relates to a method for identifying the presence of a BBB-specific protein or a fragment thereof in endothelial cells of brain capillaries (brain microvessel endothelial cells; BMEC) as well as the proteins or fragments thereof obtained by this method (BBB=blood brain barrier). The invention further relates to the genes and transcripts, respectively, obtained by said method.

The endothelial cells of cerebral capillaries form a selective permeability barrier between the blood and the brain of an organism, the so-called blood-brain barrier (BBB). Within the capillaries individual endothelial cells are arranged around the lumen and form a cylindrical tubular cavity. Tight junctions between the individual endothelial cells and other cell types associated with the endothelial cells prevent the uncontrolled passive passage of a multitude of substances through this cell layer.

For maintaining its function the brain to a high degree depends on a constant internal milieu, which is-ensured by the blood brain barrier. This also regulates the exchange of substances between the blood and the brain. Specific transporters mediate this exchange. The formation of this barrier in the endothelial cells of brain capillaries (brain microvessel endothelial cells, BMEC) is founded in the expression of specific proteins in this, as compared to other endothelial cells, highly differentiated cell type. Some proteins specific for the blood brain barrier are already known, for example, the glucose transporter GLUT-1, which is specific for the BMEC, and which ensured the energy supply of the brain.

Due to the selective permeability features of the blood brain barrier it is difficult to treat diverse diseases of the central nervous system, since numerous drugs hardly penetrate the blood brain barrier and, therefore, arrive only in low concentration had their site of action in the brain. For the development of drugs acting in the brain it would therefore be of great importance to know the mode of operation of the blood brain barrier and the proteins involved therein. In particular, it would be of relevance to obtain knowledge of that proteins, which, compared with other cell types, are produced in particularly high or particularly low extent in the endothelial cells of brain capillaries or which are produced from certain splicing variants and bear specific post-translational modifications, respectively.

The investigation of endothelial cells of brain capillaries is associated with various problems. First, for the investigation of the protein expression in the human brain there is not enough brain material available, wherein ethical reasons, among others, play a role. Further, the particular individuals, from which the brain mass originates, are normally very different as regards the genetic information. Differences, for example, arise as regards age, sex, weight, race and so on. Moreover, the material to be tested must be removed within the first hours after the death occurs, because after this period a significant alteration of the protein composition in the cells by enzymatic degradation and modification processes already takes place. In addition, previous methods for investigating the protein expression in endothelial cells of brain capillaries are afflicted with the problem that the material to be tested cannot be obtained in sufficient purity for direct investigations. Upon isolating endothelial cells of brain capillaries according to the known method usually a mixture with other cell types is obtained so that investigations of the protein expression pattern with these samples do not permit sufficient assignment exclusively to the endothelial cells of brain capillaries.

Therefore, object of the present invention is to provide a method with which BBB-specific proteins or fragments thereof can unambiguously be identified. The method shall particularly be suitable for identifying BBB-specific proteins and genes, respectively, in endothelial cells of brain capillaries. Further, the method shall be realizable simply and gently. Moreover, the method of the invention shall selectively be for proteins or fragments thereof, which are prevalently or selectively formed in endothelial cells of brain capillaries, but not in comparative tissue and related cell types, respectively. Further, proteins or fragments thereof identified with the method of the invention shall be suitable as diagnostic markers for diseases associated with a dysfunction of the blood-brain barrier. Moreover, the proteins identified with the method of the invention shall be suitable for the manufacture of medicaments for the treatment of diseases associated with a dysfunction of the blood-brain barrier.

According to the invention, the object is solved by a method for identifying the presence of a BBB-specific protein or fragment thereof in endothelial cells of brain capillaries, characterized in that a) endothelial cells of brain capillaries freshly isolated from brain are conventionally prepurified by enzymatic digestion, b) the digest obtained in step a) is treated with a lysis buffer that essentially destroys present erythrocytes and apoptotic cells and maintains at least 70% of the endothelial cells of brain capillaries in vital form, c) the product obtained in step b) is optionally purified further, d) a subtractive cDNA library is prepared from the endothelial cells of brain capillaries and a subtractive tissue, e) a cDNA subtraction is performed using one ore more differential hybridization steps, f) clones from the subtractive cDNA library are verified by differential hybridization with respect to their respective expression, g) the cDNA sequence is completed for the BBB-specific clones from the subtractive cDNA library, and h) the expression patterns of the investigated clones is compared between fresh and cultured endothelial cells of brain capillaries and, that way, the presence of BBB-specific proteins or fragments thereof is identified.

Further, the invention relates to a method for identifying the presence of a BBB-specific protein or fragment thereof in endothelial cells of brain capillaries, characterized in that a) endothelial cells of brain capillaries freshly isolated from brain are conventionally prepurified by enzymatic digestion, b) the digest obtained in step a) is treated with a lysis buffer that essentially destroys present erythrocytes and apoptotic cells and maintains at least 70% of the endothelial cells of brain capillaries in vital form, c) the product obtained in step b) is optionally purified further, d) the product obtained in step c) is solubilized in a suitable buffer, e) an isoelectric focusing is performed, f) the samples from the isoelectric focusing are separated in the second dimension according to the molecular weight, g) differential spots are identified and isolated, h) a mass spectrometric analysis is performed with the isolate of g), and i) an evaluation thereof is conducted via specific database analysis.

With the method according to the invention, BBB-specific proteins or fragments thereof can unambiguously and reliably be identified and the invention also relates to the proteins isolated with this method as well as the transcripts and genes, respectively, coding these proteins. In particular, the invention also relates to proteins having the sequences of SEQ ID NO: 5, 14, 19, 23, 27, 33, 53, isolated according to this method.

Furthermore, the invention relates to the use of the proteins and fragments thereof, respectively, identified by the method of the invention for the manufacture of agents or medicaments for diagnosis or therapy of diseases due to a dysfunction of the blood-brain barrier.

It was surprisingly found that the combination of the above processing steps allows for the unambiguous identification of BBB-specific proteins in endothelial cells of brain capillaries. The proteins isolated with the method of the invention are specific for the BBB. The proteins isolated by the method of the invention due to their specificity for the BBB have a function in and at, respectively, the BBB. This function can, for example, be a barrier function, a transport function, a function connected with the nutrient supply of the BBB, a function as a tight junction protein, an enzymatic activity etc. Thus, it is possible to specifically deduce specific functions thereof in the BBB, starting from the identification of the presence of these proteins. This opens up the possibility of entirely new therapeutical concepts being based on the fact that substances can selectively be transported through the BBB. Further, proteins identified with the method according to the invention can specifically be subject of therapeutic interventions. The method according to the invention for the first time allows the development of therapeutical concepts for diseases concerning the brain. Furthermore, the detection of alterations in proteins identified according to the described method can be employed for diagnosing diseases that are based on a dysfunction of the BBB.

The use of freshly isolated BMEC (primary cells) instead of cultivated BMEC is of particular importance in the method according to the invention. It was surprisingly found that BMEC in culture dedifferentiate very quickly, i.e. they very quickly lose their BBB features. Further, it was found that the expression of the proteins specific for the blood-brain barrier in cultivated endothelial cells of brain capillaries is strongly down-regulated and completely disappears after only a few passages, wherein no reliable isolation and identification of BMEC-specific proteins is possible. Besides, pure and vital cells must be isolated to guarantee cell specificity and prevent negative effects or effects falsifying the result via apoptosis.

In the method according to the invention the removal of brain material from the respective organism by surgery on the living organism. That way, also brain samples from the human organism, for example, can be obtained with brain surgery. Yet, the removal of the complete brain or of parts thereof from the organism, preferably immediately after death occurs, is more favourable. Preferably, the brain is to removed within a period of no more than one hour, more preferably, no more than about 30 minutes, even more preferably no more than about 15 minutes or even more preferably about 5 minutes after death occurs. The brain can be removed from any animal, for example, man, cattle, sheep, goats, horses etc. It was now found that pig brains are a good model for the human brain as regards the analysis of the endothelial cells of brain capillaries as well as the transferability of the results to men.

The pig brain, both as regards the anatomy and the morphology, is very similar to the human brain. Furthermore, sequence homologies between man and pig are generally very high on both, the protein and nucleic acid levels, so that results obtained with pig material can reliably be transferred to the man and vice versa. This is founded in that man and pig are phylogenetically more closely related than man and classical model organisms such as mouse or rat.

It was surprisingly found that a hypotonic lysis buffer does not only lyse erythrocytes but also generally causes dead and apoptotic cells to burst through hypotonic shock. The lysis buffer to be used in the method according to the invention preserves at least 70%, preferably 80%, more preferably 90%, even more preferably 95% of the endothelial cells of brain capillaries in vital form. Furthermore, the lysis buffer must be non-toxic and must have a pH value in the physiological range. The hypotonic buffer used according to the invention should have an ionic strength of 0.1-0.2 M, contain monovalent and bivalent anions and cations, respectively, and buffer in a pH range of 7.0-8.0. All substances contained must be non-toxic for the cells so that healthy cells are not damaged in the buffer for short time. Preferably, the hypotonic buffer having an ionic strength of 0.1-0.2 M contains sodium, potassium, ammonium, calcium, magnesium, chloride and sulphate ions as well as glucose and buffers in a pH range of 7.0-8.0. This allows for the selective enrichment of vital endothelial cells of brain capillaries from a mixture of erythrocytes and other cells of varying vitality. The buffer used according to the invention preferably has the following composition at a pH value of 7.5:

| ion/substance | min. conc. [mM] | max. conc. [mM] |
|---|---|---|
| Na⁺ | 30.0 | 60.0 |
| K⁺ | 5.0 | 7.5 |
| NH₄⁺ | 80.0 | 100.0 |
| Ca²⁺ | 1.0 | 2.0 |
| Mg²⁺ | 6.0 | 9.0 |
| Cl⁻ | 125.0 | 175.0 |
| HCO₃⁻ | 4.5 | 6.5 |
| H₂PO₄⁻ | 0.5 | 2.5 |
| SO₄²⁻ | 0.3 | 0.6 |
| HPO₄²⁻ | 0.4 | 0.7 |
| Glucose | 1.5 | 3.0 |

More preferably, the lysis buffer used has the following composition:

| | | | | | |
|---|---|---|---|---|---|
| NaCl | 30 | mM | to | 50 | mM |
| KCl | 4.5 | mM | to | 5.5 | mM |
| NH₄Cl | 80 | mM | to | 100 | mM |
| CaCl₂ | 1.0 | mM | to | 2.0 | mM |
| MgCl₂ | 0.6 | mM | to | 0.8 | mM |
| MgSO₄ | 0.3 | mM | to | 0.6 | mM |
| NaHCO₃ | 4.5 | mM | to | 6.5 | mM |
| NaH₂PO₄ | 0.2 | mM | to | 0.45 | mM |
| Na₂HPO₄ | 0.4 | mM | to | 0.65 | mM |
| KH₂PO₄ | 0.1 | mM | to | 0.15 | mM |
| Glucose | 1.5 | mM | to | 3.0 | mM |

Particularly preferred, the buffer has the following composition:

| | | |
|---|---|---|
| NaCl | 39 | mM |
| KCl | 5.1 | mM |
| NH₄Cl | 88 | mM |
| CaCl₂ | 1.6 | mM |
| MgCl₂ | 0.69 | mM |
| MgSO₄ | 0.46 | mM |
| NaHCO₃ | 5.6 | mM |
| NaH₂PO₄ | 0.33 | mM |
| Na₂HPO₄ | 0.53 | mM |
| KH₂PO₄ | 0.12 | mM |
| Glucose | 2.24 | mM |

Normally, such lysis buffers are used for the isolation of lymphocytes and RNA from lymphocytes, respectively, by first lysing the erythrocytes at this. Up to now, neither the composition of the buffer used according to the invention nor the use of such buffer for the lysis of apoptotic cells has been reported. The selective lysis of apoptotic cells is of significant importance at the method according to the invention in order to enrich BBB-specific transcripts without simultaneously enriching transcripts of genes being more strongly expressed during apoptosis. In other methods for isolating cells the problem of apoptosis is avoided in that the isolated cells are cultured. Endothelial cells of brain capillaries, however, alter their features in culture, leading to an altered gene expression pattern. Therefore, the method for cell preparation via the final lysis step according to the invention for the first time allows and specifically allows the isolation of sufficient amounts of fresh endothelial cells of brain capillaries.

After the removal of the brain from the organism the brain is practically transferred in a suitable buffer and transported as quickly as possible, cooled on ice into the lap for further processing. The endothelial cells of brain capillaries to be isolated are primarily located in the grey matter. Advantageously, prior to further purification of the cells the grey matter is mechanically dissected from the remaining brain parts. For this, at first, meninx is peeled off and the grey matter is abraded, reduced to small pieces and transferred into a suitable medium. A suitable medium, for example, is M199 medium (Gibco/BRL, Grand Island, N.Y.) or Earle's buffer. Prior to further purification it is practical to determine the mass of the grey matter obtained.

| | | | |
|---|---|---|---|
| Earle's buffer: | NaCl | 117.2 | mM |
| (pH 7.3) | KCl | 5.3 | mM |
| | NaH₂PO₄ × 2H₂O | 1.0 | mM |
| | MgSO₄ × 7H₂O | 0.81 | mM |
| | CaCl₂ × 2H₂O | 1.8 | mM |
| | Glucose × H₂O | 5.6 | mM |

According to the invention the pre-purification of the endothelial cells of brain capillaries occurs via digesting the brain substance in at least two enzymatic steps following each other. In a first enzymatic step the brain substance is digested with the enzyme dispase. Dispase digestion causes the disintegration of the nerve tissue. An amount of 5 mg dispase per gram grey matter has proven to be particularly suitable. Dispase digestion is practically carried out in M199 medium, however, also other media and buffers are suitable for this reaction. A correspondingly prepared dispase solution is added to the sample of the grey matter and the suspension is incubated at 37° C. under stirring. Incubation times of two to four hours, preferably about three hours, have proven to be particularly advantageous. The enzyme concentrations, the solvents and media used, respectively, and the incubation time in each case are to be selected such that as much of the material surrounding and binding, respectively, the brain capillaries is degraded and disintegrated, respectively. However, at the same time, the conditions are to be adjusted such that a part as small as possible of the endothelial cells of brain capillaries to be isolated are affected and killed, respectively, in the respective enzymatic step and that the cells are exposed to as small a strain as possible.

At this, it is essential that resulting shearing forces are kept as small as possible. This is, for example, achieved in that the enzymatic digest of the brain mass is mixed slowly and continuously in spinner bottles.

After the dispase digest in a first purification step the brain capillaries are attained via centrifugation in dextran solution. For this purpose, methods known from the prior art can be employed. It has proven to be particularly suitable to mix an amount of the cell suspension from the dispase digest with the equal amount of a 15% dextran solution, to shake for 10 minutes and to centrifuge for about 10 minutes at 10° C. at 8,650×g in a fixed angle rotor. After centrifugation the supernatant is removed and the sediment subjected to the second enzymatic step.

In the second enzymatic step the sediment of the centrifugation is digested with collagenase D. Collagenase D, among other, dissolves the basement membrane. One or more protease inhibitors are practically added to the second enzymatic step. For this purpose, the protease inhibitor Na-p-tosyl-L-lysine-chloromethylketone (TLCK) is particularly suitable;. The second enzymatic step is practically performed under stirring at 37° C. for about one hour. It has also proven to be particularly suitable to employ one or more DNAses such as benzonase in the second enzymatic step. Through this, during the digest of dead cells released DNA is degraded, which otherwise increases the viscosity of the suspension.

After the second enzymatic step, a second purification step via centrifugation in a Percoll density gradient is carried out. The density gradient is prepared in that for example 9.91 ml Percoll, 0.72 ml 10-fold concentrated M199 medium and 19.37 ml Earle's buffer are mixed and centrifuged for one hour at 37,200×g at 4° C. in a fixed angel rotor in an ultra centrifuge. The cell suspension from the second enzymatic step is washed via multiple centrifugation at low velocity, taking off the supernatant and resuspending the centrifugational sediment, e.g. freed from the added enzymes. After the last centrifugation step the sediment is taken up in a small amount of liquid such as 6 ml M199 medium, and applied on the prepared Percoll density gradient and centrifuged in the swing-out rotor in the ultracentrifuge at 1,400×g, 4° C., for ten minutes. The Percoll density gradient centrifugation causes a separation of the suspended cell material according to its density, wherein usually three discrete bands occur. A first upper band, having the lowest density, contains cell debris and cell fragments, respectively. A second intermediate band contains the endothelial cells of brain capillaries to be isolated, among others. In a third lower band having the highest density erythrocytes among others collect.

The second band containing the endothelial cells of brain capillaries is isolated and subjected to further purification according to the invention. Isolation can be carried out via taking off the band with the aid of a needle or, preferably, by pipetting off.

Next to the endothelial cells of brain capillaries the material of the second band obtained from the Percoll density gradient centrifugation contains a plurality of other cell types, primarily erythrocytes and apoptotic cells. Up to now, it has not been possible to separate these contaminating cells sufficiently from the endothelial cells of brain capillaries under gentle conditions. Surprisingly, it was now found that this problem can be solved if further purification of the endothelial cells of brain capillaries is carried out with a lysis buffer commonly used for the isolation of lymphocytes, wherein the composition of the lysis buffer, the duration of the treatment and the treatment temperature are selected such that erythrocytes and apoptotic cells are essentially completely destroyed and a great portion of the endothelial cells of brain capillaries survives. The advantages and features of this buffer were set forth above.

A lysis buffer containing the following components has proven to be suitable according to the invention.

| | | | | | |
|---|---|---|---|---|---|
| NaCl | 30 | mM | to | 50 | mM |
| KCl | 4.5 | mM | to | 5.5 | mM |
| NH₄Cl | 80 | mM | to | 100 | mM |
| CaCl₂ | 1.0 | mM | to | 2.0 | mM |
| MgCl₂ | 0.6 | mM | to | 0.8 | mM |
| MgSO₄ | 0.3 | mM | to | 0.6 | mM |
| NaHCO₃ | 4.5 | mM | to | 6.5 | mM |
| NaH₂PO₄ | 0.2 | mM | to | 0.45 | mM |
| Na₂HPO₄ | 0.4 | mM | to | 0.65 | mM |
| KH₂PO₄ | 0.1 | mM | to | 0.15 | mM |
| Glucose | 1.5 | mM | to | 3.0 | mM |

A lysis buffer having the following composition is particularly suitable:

| | | |
|---|---|---|
| NaCl | 39 | mM |
| KCl | 5.1 | mM |
| NH₄Cl | 88 | mM |
| CaCl₂ | 1.6 | mM |
| MgCl₂ | 0.69 | mM |
| MgSO₄ | 0.46 | mM |
| NaHCO₃ | 5.6 | mM |
| NaH₂PO₄ | 0.33 | mM |
| Na₂HPO₄ | 0.53 | mM |
| KH₂PO₄ | 0.12 | mM |
| Glucose | 2.24 | mM |

After adding the lysis buffer the suspension is mixed and repeatedly washed via centrifugation at low velocity and resuspension in a suitable medium and buffer, respectively, such as M199 or Earle's buffer. The purified endothelial cells of brain capillaries collect in the centrifugate.

The purified endothelial cells of brain capillaries can now be processed via two different routes in order to identify the presence of BBB-specific proteins or fragments thereof. That way, via the proteomics approach, on the one hand, or the genomics approach on the other hand, in each case different proteins, fragments thereof and transcripts, respectively, can be identified and isolated. In the following, both approaches are described in further detail.

The following figures further illustrate the subject matter of the present invention:

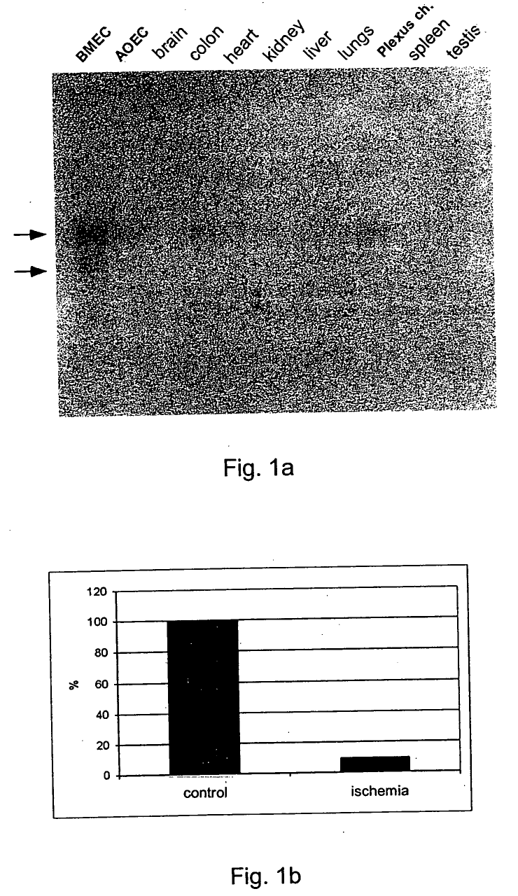

FIG. 1a: Northern blot analysis of Itm2A

FIG. 1b: Expression of Itm2A in BMEC under ischemia

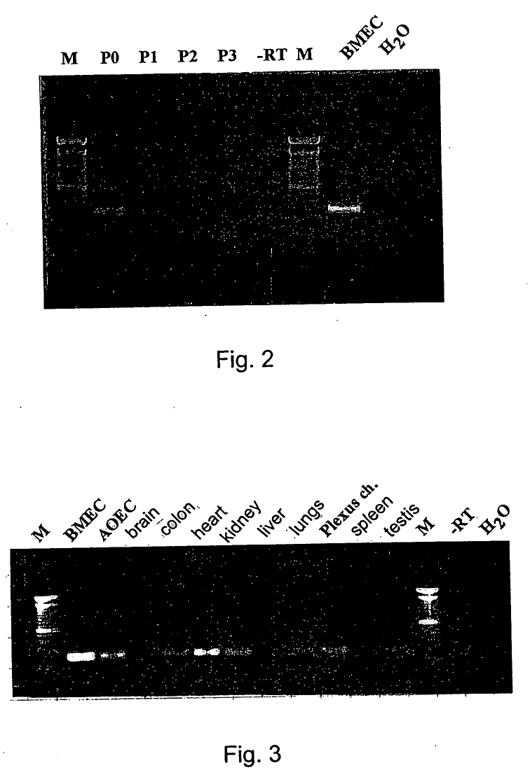

FIG. 2: Expression pattern of Itm2A in cultivated BMEC (M:100 bp marker)

FIG. 3: Expression pattern of S231 (M:100 bp ladder)

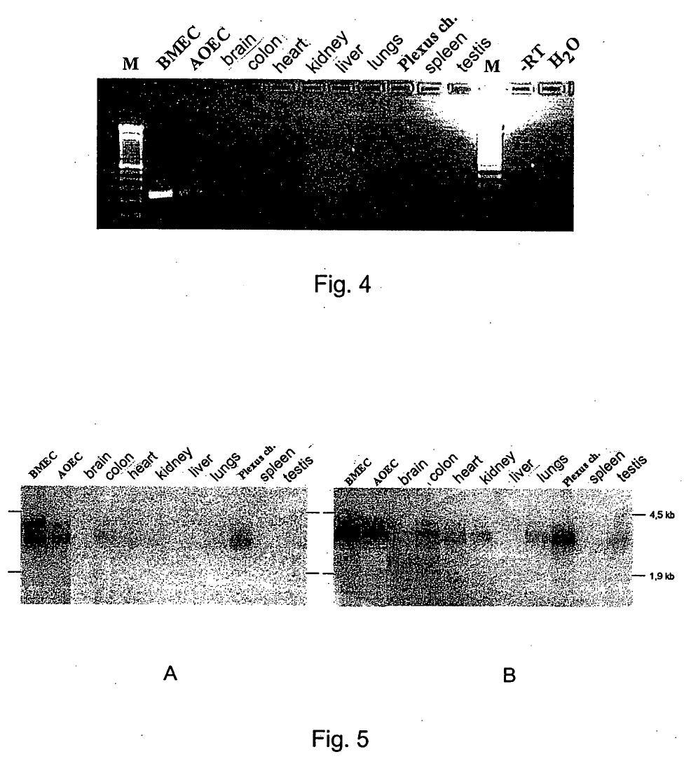

FIG. 4: Expression pattern of ssEMP1 (M:100 bp ladder)

FIG. 5: Northern blot analysis hybridized with S231 (A) and EMP1 (B), respectively, as probe

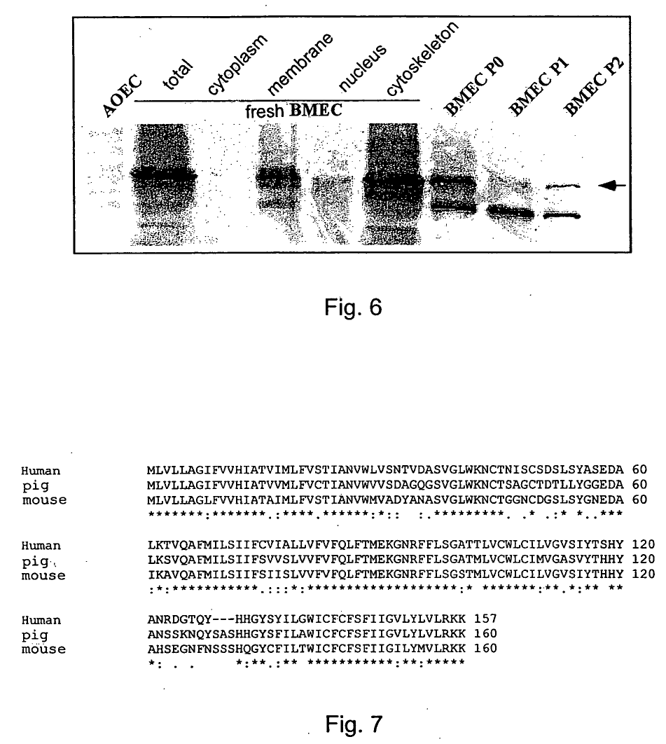

FIG. 6: Western blot analysis of S231

FIG. 7: Homology comparison of human and murine EMP1 as well as porcine S231. The membrane domain is highlighted pale, the N-glycosylation site light-grey.

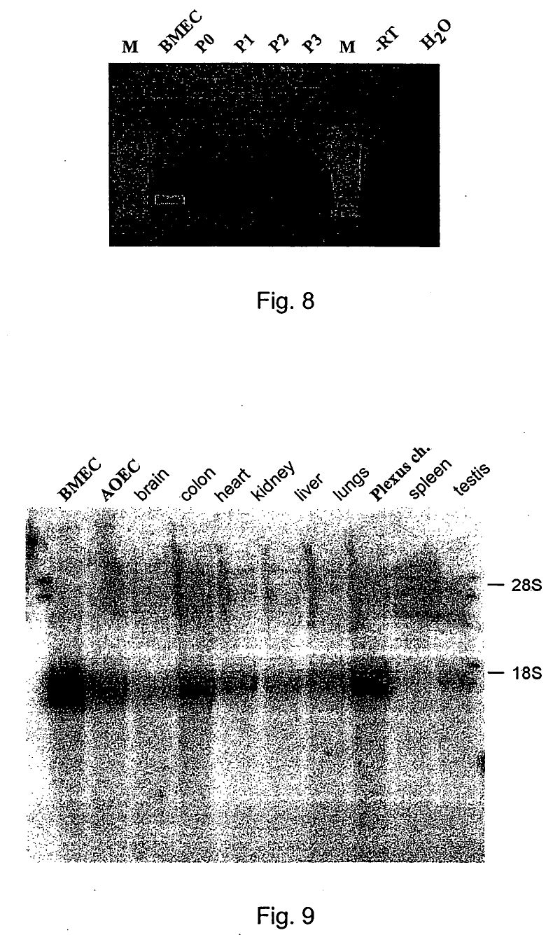

FIG. 8: Expression pattern of S231 in cultivated cells (M:100 bp marker)

FIG. 9: Northern blot, hybridized with full-length FLJ13448/S012 as a probe

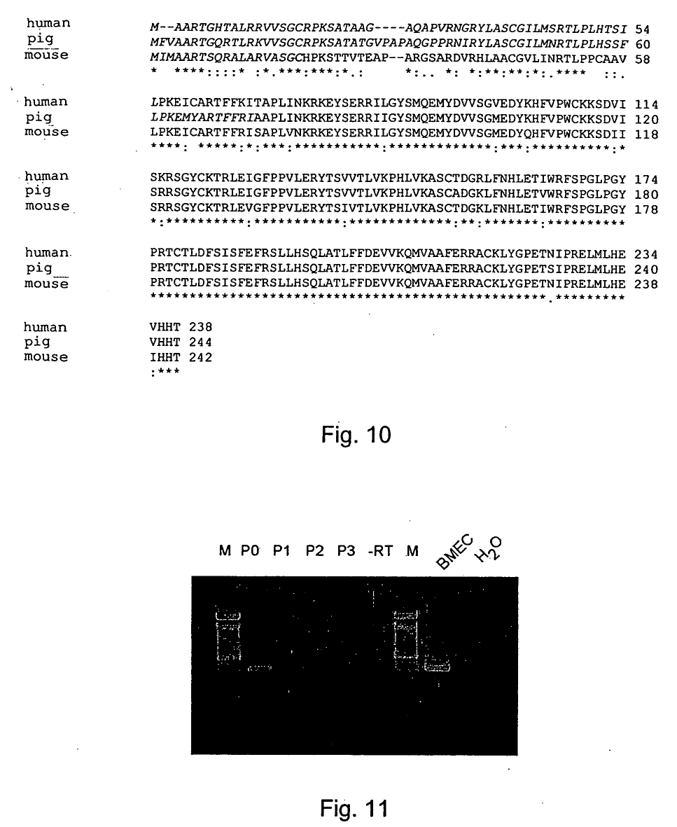

FIG. 10: Homology comparison of human, murine and porcine FLJ13448/S012. In each case, the peptides which serve as signal peptides, and which are cleaved off, are depicted in italics.

FIG. 11: Expression pattern of porcine FLJ13448/S012 in cultivated cells (M:100 bp marker)

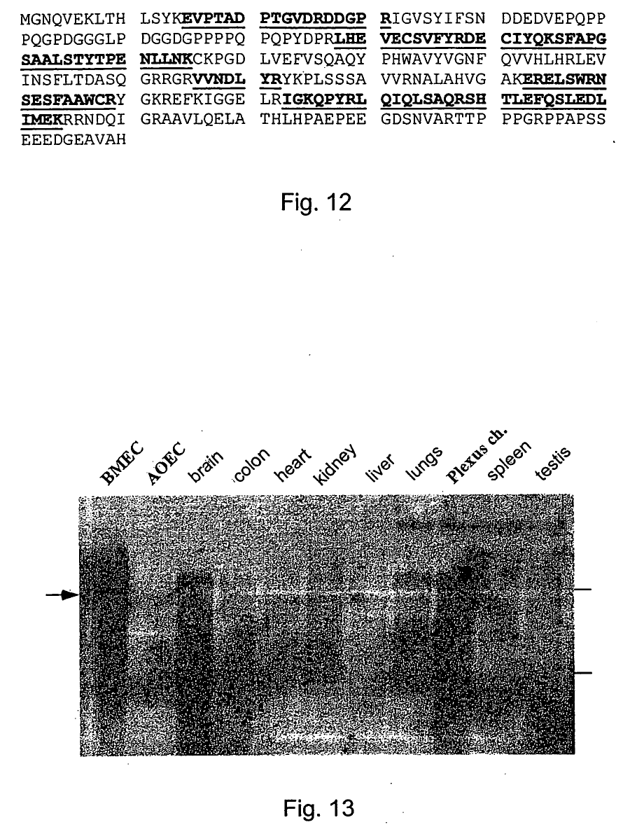

FIG. 12: NSE2 amino acid sequence of the human protein. The peptides identified in mass finger printing are marked by the bold underlined font.

FIG. 13: Northern blot of NSE2, hybridized with SEQ ID NO: 22 as probe

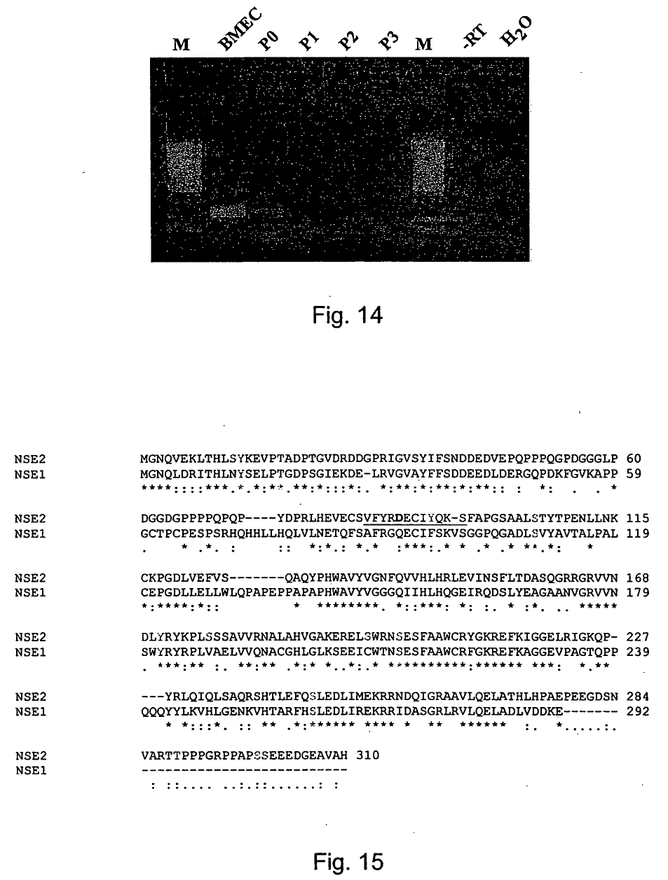

FIG. 14: Expression pattern of NSE2 in cultivated cells (M:100 bp marker)

FIG. 15: Homology comparison of human NSE2 and NSE1. Potential phosphorylation sites are depicted in pale font. A potential tyrosine kinase domain (ProSite Pattern Match PS00109) is underlined, wherein the active residue is depicted in bold.

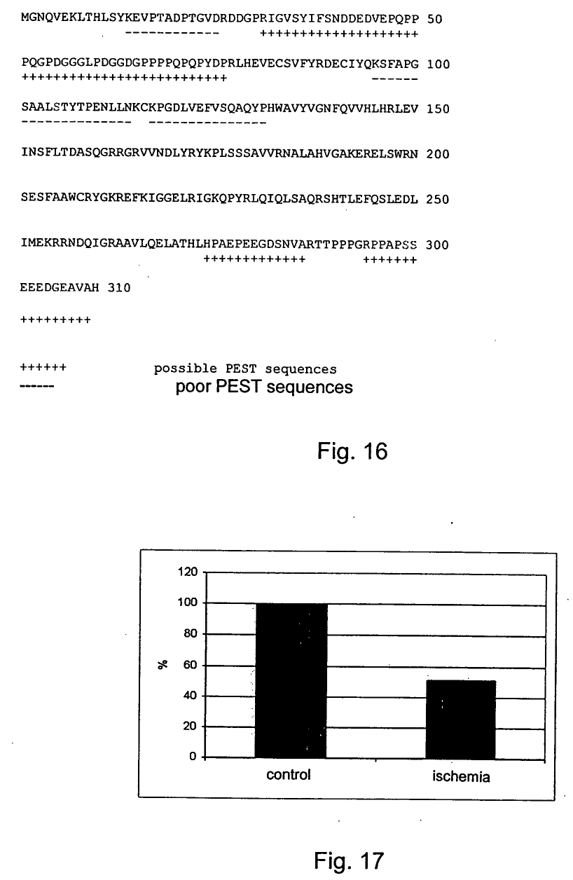

FIG. 16: Distribution of PEST domains in NSE2. PEST sequences are Pro, Glu, Ser and Thr rich regions in proteins, which are responsible for a short half-life of such proteins in the cell in that they control the ubiquitinylation of said proteins. Phosphorylation of Ser or Thr residues in the PEST regions (pale) is important for the recognition and processing via the ubiquitin proteasome pathway.

FIG. 17: Expression of NSE2 in BMEC under ischemia

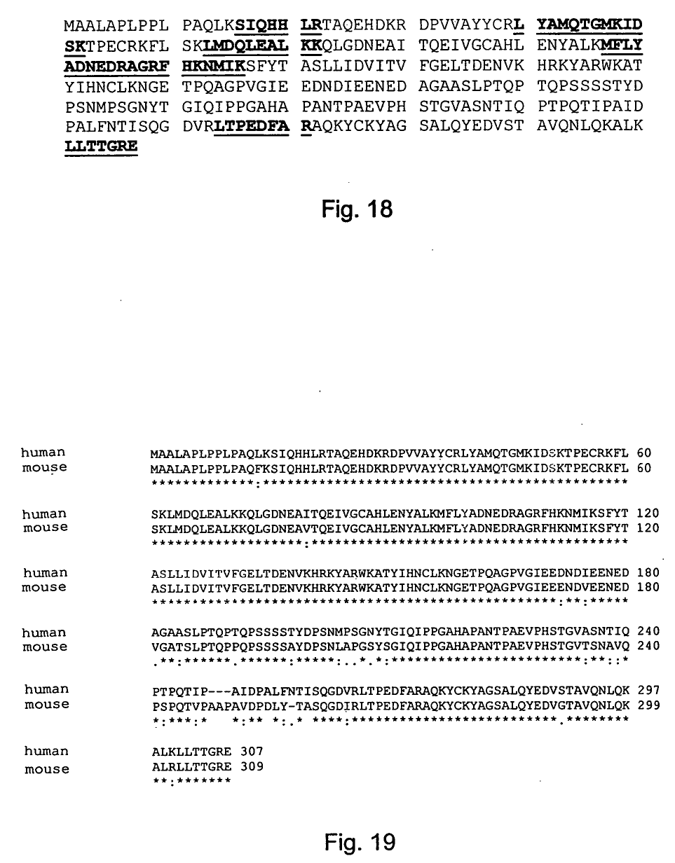

FIG. 18: Amino acid sequence of the human protein DRG-1 (CAB66619). The peptides identified in mass finger printing are marked by the bold underlined font.

FIG. 19: Homology comparison of human and murine DRG-1 shows 90% identity and 94% homology, respectively. Potential phosphorylation sites, a nonconserved potential glycosylation site and the transmembrane domain are depicted in pale font. The N-terminus is localised intracellularly.

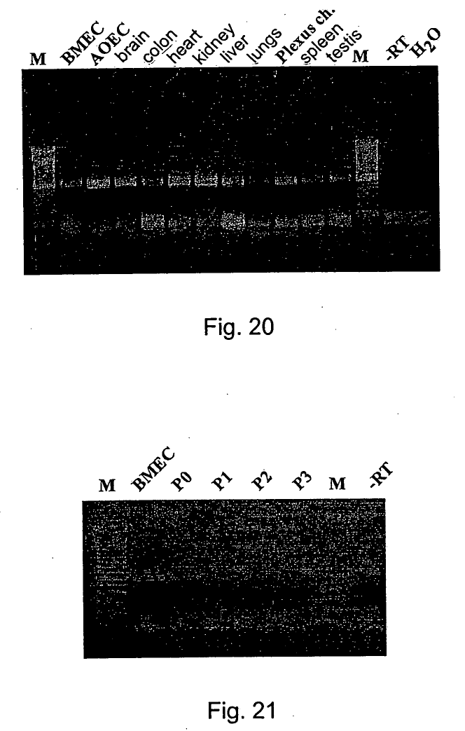

FIG. 20: Expression pattern of DRG-1 (M:100 bp marker)

FIG. 21: Expression pattern of DRG-1 in cultivated cells (M:100 bp marker)

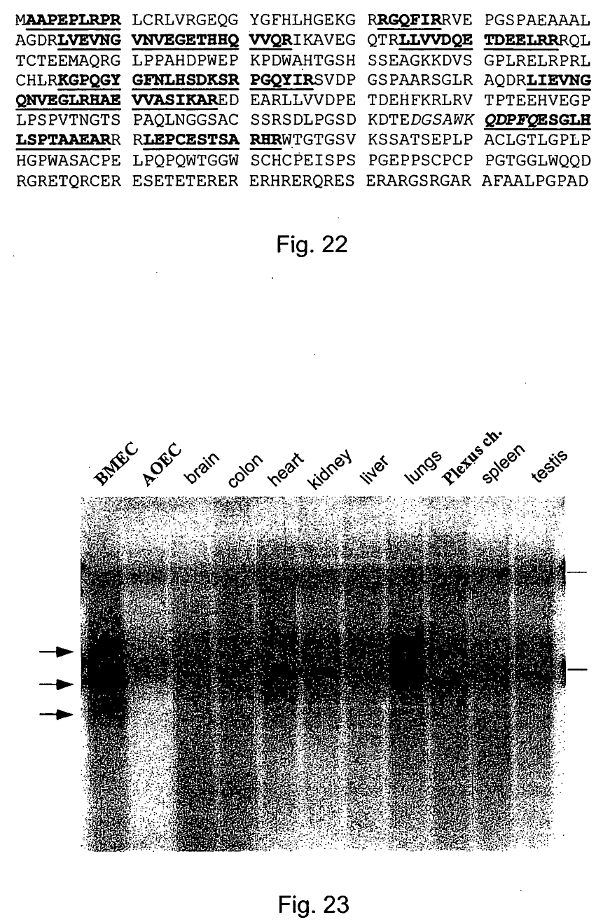

FIG. 22: TKA-1 amino acid sequence of the human protein. The peptides identified in mass finger printing are marked by the bold underlined font.

FIG. 23: Northern blot, hybridized with ssTKA-1.ctg as probe

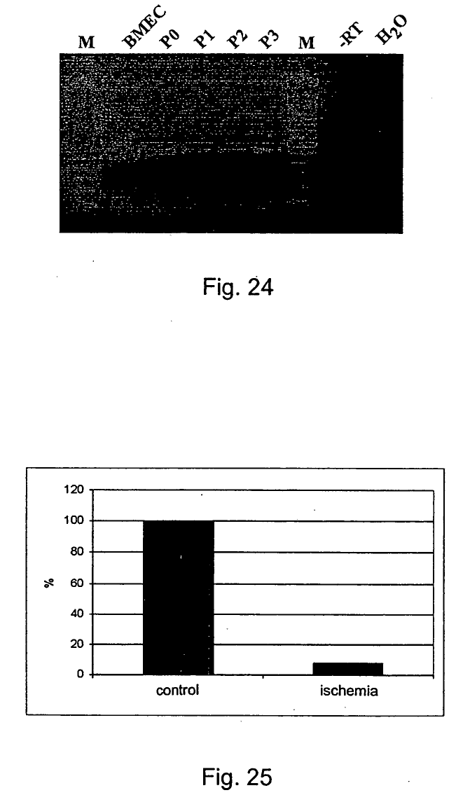

FIG. 24: Expression pattern of TKA-1 in cultivated cells (M:100 bp marker)

FIG. 25: Expression of TKA-1 in BMEC under ischemia

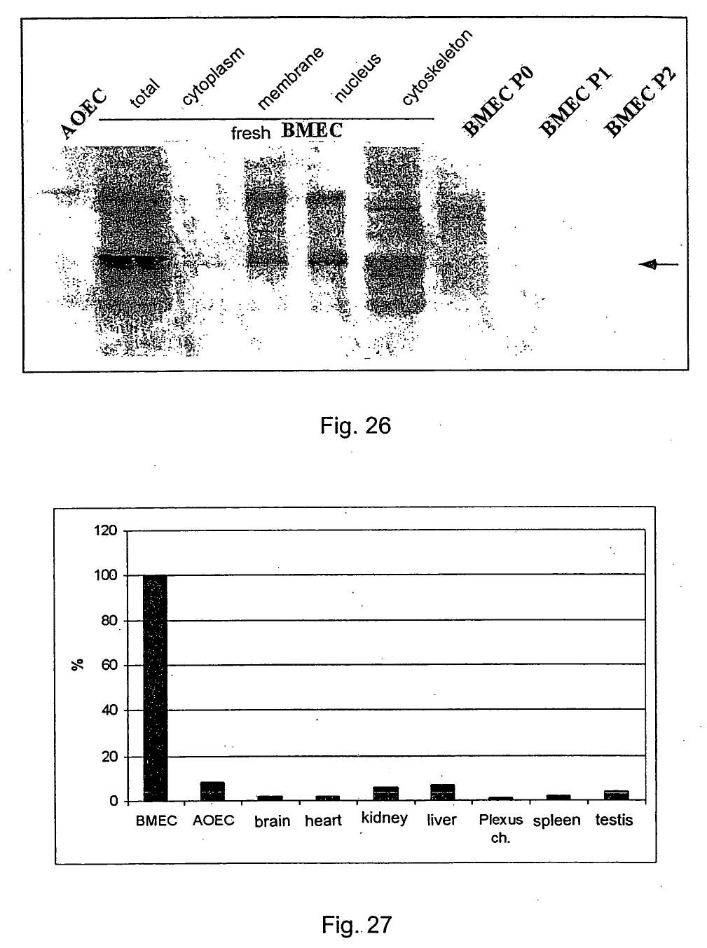

FIG. 26: Western blot analysis of TKA-1

FIG. 27: Expression pattern of S064

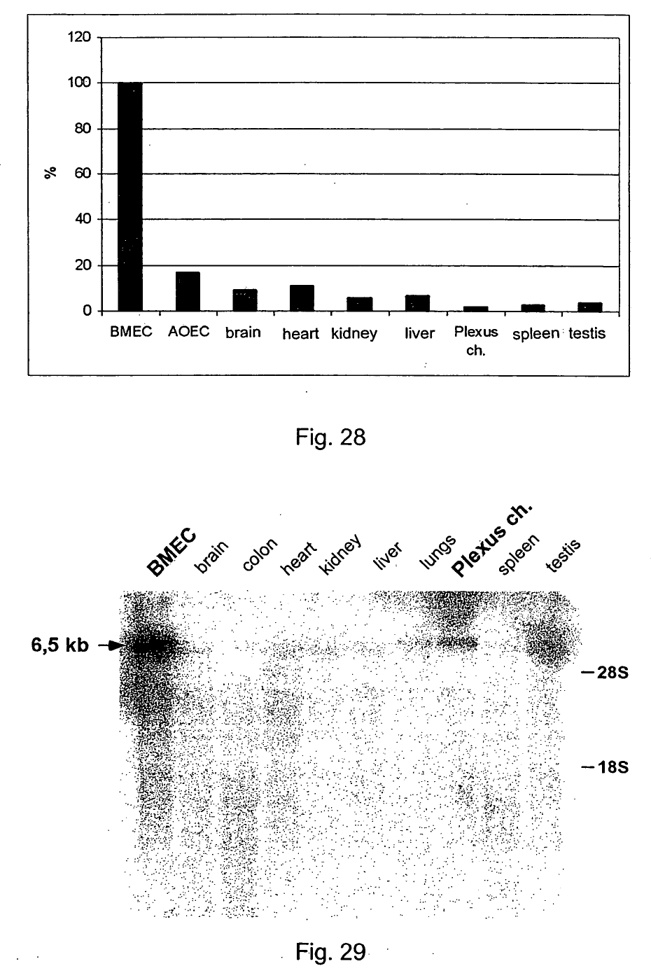

FIG. 28: Expression pattern of ARL8

FIG. 29: Multiple tissue blot, hybridized with S064 as a probe

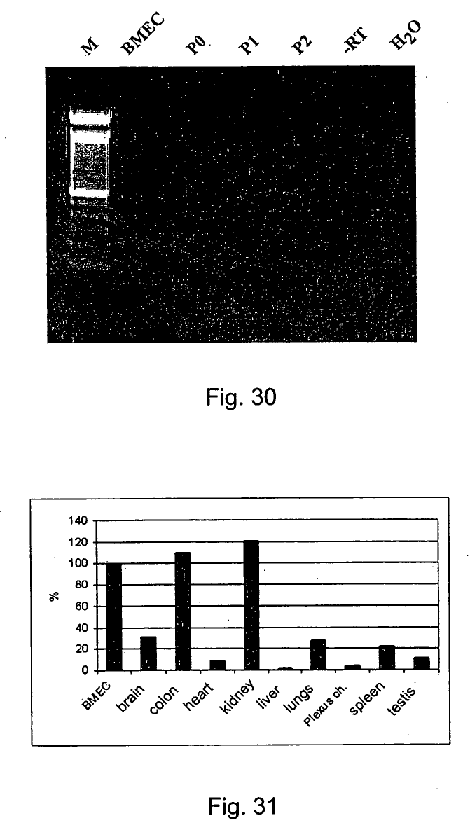

FIG. 30: Expression of S064/ARL8 in cultivated BMEC

FIG. 31: Expression pattern of 5G9

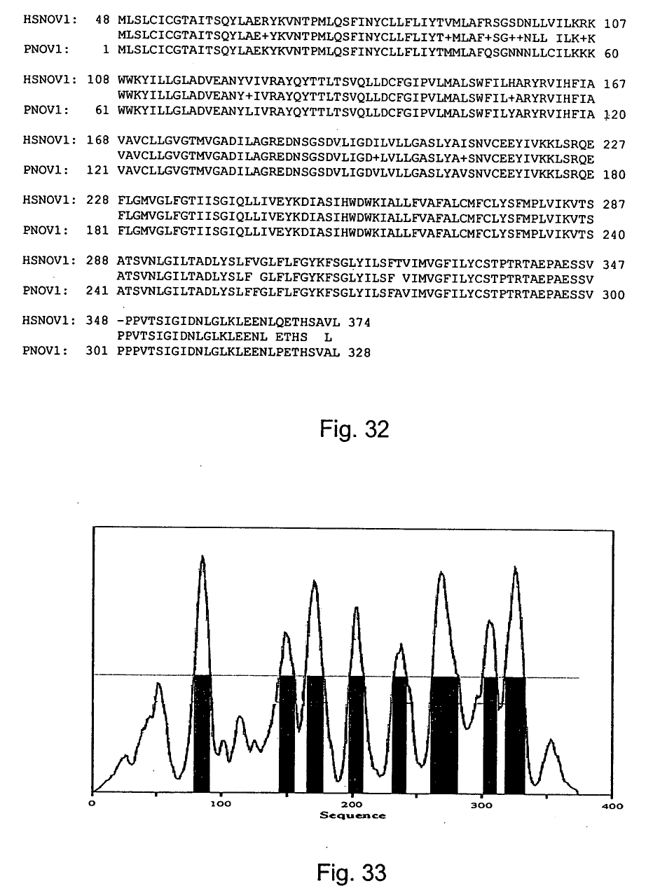

FIG. 32: Homology comparison between HSNOV1 and PNOV1

FIG. 33: Prediction of transmembrane domains within the sequence of the protein HSNOV1

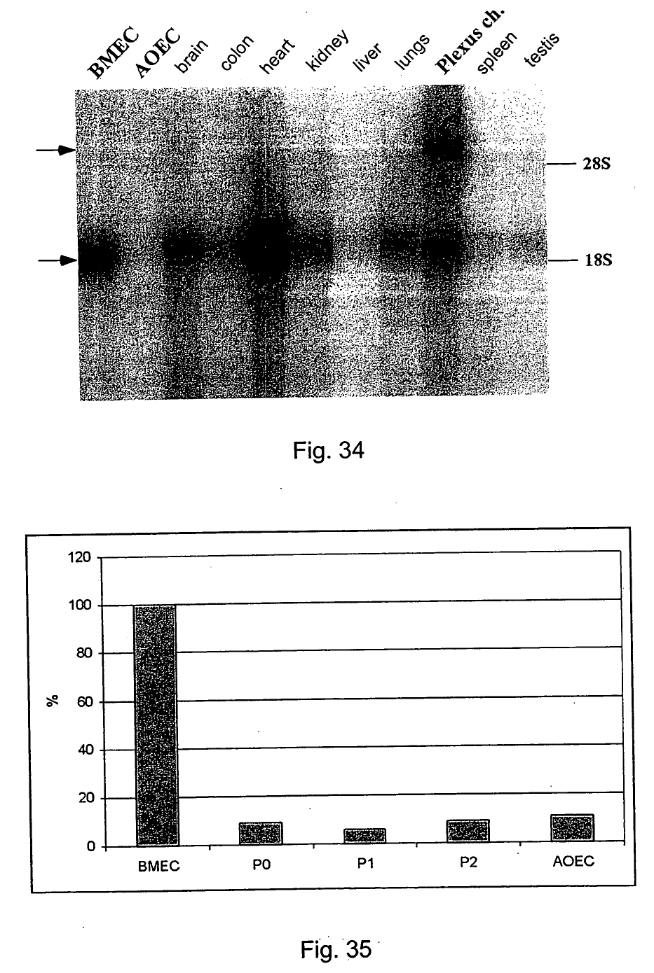

FIG. 34: Multiple tissue blot, hybridized with 5E7 as probe

FIG. 35: Expression pattern of TSC-22 in cultivated BMEC

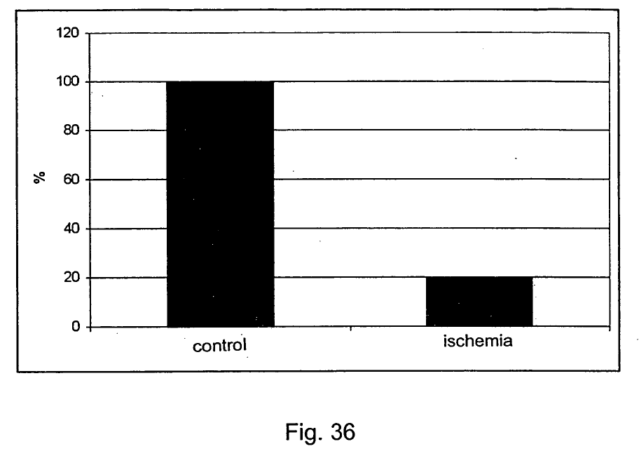

FIG. 36: Reduced expression rate of TSC-22 in BMEC upon ischemia

### IDENTIFICATION OF BBB-SPECIFIC PROTEINS VIA 2D DIFFERENTIAL GEL ELECTROPHORESIS

By the direct two-dimensional comparison of the gene products a complete picture of the endothelial cells of brain capillaries can be obtained. According to the invention, a comparative tissue is used in all electrophoreses. The comparative tissue is a tissue allowing a selective identification of transcripts and proteins, respectively, specific for the blood-brain barrier. In principle, any endothelial cells, for example, macro- and microvascular endothelial cells of the same tissue or also endothelial cells from other tissues, e.g. heart, lungs, kidney, liver, aorta etc. can be used as comparative tissue. Also de-differentiated BMEC attained from culture can be used. However, it is preferable to use another endothelial cell type as comparative tissue vis-a-vis endothelial cells of brain capillaries. Preferably used are endothelial cells from aorta, which exhibit no barrier function. This, additional has the advantage that microvessels can be compared with macrovessels. Furthermore, also other microvascular endothelial cells can be used. Also suitable as comparative tissue are endothelial cells of brain capillaries cultivated under other conditions, e.g. under other conditions as regards pH value, growth matrix, growth factor such as cytokines. The physiological significance of the identified protein follows from the known features of endothelial cells of brain capillaries vis-a-vis the respective comparative tissue. Two defined cell types are preferably used according to the invention: Freshly isolated BMEC as the cell type with barrier function and endothelial cells from aorta, which like BMEC are also endothelial cells, yet without exhibiting barrier function. In particular, by using pig tissue it is possible for the first time to prepare such a detailed proteome map of these cells.

Sample Preparation

At first, the vitality of the prepared cells and the portion of the erythrocytes contained in the preparation needs to be determined. For the determination of the vitality 20 μl of the suspended cells are taken and added with 4 μl fluorescein diacetate working solution (24 μM in Earle's buffer) and 2 μl propidium iodide working solution (70 μM in Earle's buffer). The suspension is mixed and incubated for 10 min. at 37° C. The cells are documented under a fluorescent microscope and the ratio of vital cells to damaged cells is determined. Living cells can be recognized due to a green fluorescence (excitation 450 nm and emission 515 nm), damaged cells, however, due to a red fluorescence localized to the nucleus (excitation 488 nm and emission 615 nm). The portion of erythrocytes is determined by addition of 20 μl benzidine working solution (15 mM benzidine hydrochloride, 12% (v/v) acidic acid, 2% (v/v) H₂O₂) to 20 μl cell suspension. The sample is mixed and incubated for five min. at 25° C. Then a drop of the cells was pipetted onto a microscope slide and covered with a cover slip. In this test, erythrocytes appear by the attachment of blue crystals in the transmission microscope. The ratio of endothelial cells to erythrocytes is determined by counting. Cells can be used for the following two-dimensional gel electrophoresis upon a vitality ratio of 95% vital cells and upon an erythrocyte contamination of less than 10%.

The wet weight of the freshly isolated sedimented cells is determined and it is carefully resuspended with the five-fold volume (e.g. 100 mg cells with 500 μl buffer) buffer A pH 6.8 (10 mM PIPES, 100 mM NaCl, 3 mM MgCl₂, 300 mM saccharose, 5 mM EDTA, 1 mM PMSF, 150 μM digitonin). Then, the cell suspension was incubated on ice for 20 min. under slight shaking. Subsequently, a centrifugation (480 g, 4° C., 10 min) is carried out in order to sedimate the cells. The supernatant is removed and stored at −20° C. until further use.

The sediment is resuspended again in the five-fold volume of the original wet weight in buffer B pH 7.4 (10 mM PIPES, 100 mM NaCl, 3 mM MgCl₂, 300 mM saccharose, 5 mM EDTA, 1 mM PMSF, 0.5% (v/v) Triton X-100) and incubated for 30 min. under rigorous shaking on ice. Then, the sample is sedimented for 10 min. via centrifugation (5,000 g, 4° C.), the supernatant is withdrawn and stored at −20° C. until further use.

Now, the sediment is resuspended in 1.7-fold of the original wet weight in buffer C pH 7.4 (10 mM PIPES, 10 mM NaCl, 1 mM MgCl₂, 1 mM PMSF, 1% (v/v) TWEEN-40, 0.5% (w/v) desoxycholate), transferred to a Dounce homogeniser and disrupted with five movements. Then, the sample is transferred to a 2 ml reaction tube again and incubated for 1 min in the ultrasonic bath. Then, the sample is sedimented by centrifugation (6,780 g, 4° C., 10 min.) and the supernatant-is stored at −20° C. until further use.

The sediment is to be resuspended in 200-500 1 buffer pH 8.0 (50 mM Tris, 1 mM MgCl₂) dependent on its size and is shock-frozen in nitrogen. Thereafter, the sample is thawed in the ultrasonic bath and subsequently incubated at 37° C. with 5-10 μl benzonase (25 U/μl) until a homogenous, no longer viscose liquid forms. Then, the 7-fold volume of a 5% (w/v) STS solution is added and the sample is heated to 90° C. for 20 min. followed by 10 min. centrifugation (7,000 g, 20° C.) for removing insoluble components. The supernatant was withdrawn and stored at −20° C. until further use. A possibly present sediment is discarded.

The supernatants are thawed, proportionally combined and mixed. In order to remove the detergents contained in the sample, the sample is mixed with the 100% acetone (stored at −30° C.) in a ratio of 20 to 80. After thorough mixing the precipitation is incubated for at least 1 h at −30° C. Then the precipitated proteins are sedimented for 15 min. at 10,000 g and 4° C. The supernatant is decanted and discarded.

Subsequently, the sediment is washed with 80% (v/v) acetone (−30° C. cold) and incubated again at −30° C. After new centrifugation (15 min., 10,000 g, 4° C.) the supernatant is discarded and sediment is resuspended in the smallest amount of solubilisation buffer I (7 M urea, 2 M thio-urea, 4% (w/v) CHAPS) or II (8 M urea, 4% (w/v) CHAPS) possible, and the protein content of the samples is determined. Regarding this, for the protein determination 1 part Rotiquant (Roth) and 4 parts bi-distilled water are mixed to a ready working solution and insoluble components are removed over a folded filter. For the calibration curve dilutions of bovine serum albumin (BSA) in solubilisation buffers I and II, respectively, are prepared. At this, concentrations of 0.2 mg/ml, 0.4 mg/ml, 0.6 mg/ml, 0.8 mg/ml and 1.0 mg/ml were adjusted for the calibration solutions. 20 μl each of the calibration solutions, the sample and the reference (solubilisation buffer I or II) are placed in a 1.5 ml reaction container and are added with 1 ml of the Rotiquant working solution. The respective reaction container is mixed by immediate reversing. Then, the sample is incubated at 25° C. for a period of 20 min. After transferring the sample in a 1 ml cuvette the absorption at 560 nm is measured in a spectrophotometer. The protein content of the samples can be determined by preparing a calibration curve.

The remaining supernatants of the samples are stored at −8° C. until further use.

Isoelectric Focusing

For 12 focusing gels 2.5 mg sample, dissolved in 4.5 ml solubilisation buffer I or II (for pH gradient 4.5-5.5), are added with 1.125 ml focussing buffer I (7 M urea, 2 M thiourea, 4% (w/v) CHAPS, 91 mM DTT, 2.5% IPG buffer) and II (8 M urea, 4% (w/v) CHAPS, 91 mM 2.5% IPG buffer; for pH gradient 4.5-5.5), respectively, treated for 1 min in the ultrasonic bath and then centrifuged for 5 min. in the table centrifuge (20.000 g). For the pH gradients used (3.5-4.5; 4.0-5.0; 4.5-5.5; 5.0-6.0; 5.5-6,7; 6.0-9.0), which are used as 24 cm long gels (Immobiline DryStrip; Amersham Biosciences), the appropriate IPG buffers are employed.

Subsequently, 450 μl of each sample are pipetted in the rehydration device and the Immobiline DryStrip focusing gel is placed gelside down without air bubbles onto the solution by means of two pairs of tweezers. The gel is overlaid with paraffin oil. The rehydration time is at least 12 h up to 16 h maximally. At the pH gradient 6.0-9.0, at which the sample is applied via cup loading, instead of the sample, the gel strip is rehydrated with the respective mixture of solubilisation buffer I (4.5 ml) with the respective focusing buffer I (1.125 ml). After rehydration each gel strip is transferred to a 24 cm stripholder, and, additionally, in the case of cup loading. The sample cup is directly in front of the cathode. The sample with 200 μg protein each is pipetted in the sample cup and, together with the Immobiline DryStrip gel overlaid with paraffin oil.

The electrode strips which are wetted with bi-distilled water are positioned at the respective gel ends. Then, the electrodes are placed on these strips. Thereafter, six loaded stripholders each are focused in ETTAN IPGphor focusing apparatus (Amersham Biosciences) which corresponds to the pH gradient (see Table 1). After completing the focusing the strips are removed with tweezers and stored at −80° C. until further use.

TABLE 1
Programmes for the isoelectric focusing

| Programme 1 | | | | |
|---|---|---|---|---|
| S1 | 500 V | linear gradient | 1 | h |
| S2 | 500 V | step gradient | 1 | h |
| S3 | 1000 V | step gradient | 1 | h |
| S4 | 8000 V | linear gradient | 1 | h |
| S5 | 8000 V | step gradient | 88 | kVh |
| Programme 2 | | | | |
| S1 | 500 V | linear gradient | 0.5 | h |
| S2 | 500 V | step gradient | 0.5 | h |
| S3 | 1000 V | linear gradient | 0.5 | h |
| S4 | 1000 V | step gradient | 0.5 | h |
| S5 | 4000 V | linear gradient | 1.0 | h |
| S6 | 4000 V | step gradient | 0.5 | h |
| S7 | 8000 V | linear gradient | 0.5 | h |
| S8 | 8000 V | step gradient | 105 | kVh |

Programme 1 is used for the pH gradients 3.5-4.5, 4.0-5.0, 4.5-5.5, 5.0-6.0 and 5.5-6.7 whereas programme 2 is used for gels with a pH gradient of 6.0-9.0.

SDS Electrophoresis

The necessary SDS polyacrylamide gels for the second dimension having a concentration of acrylamide of 12.5% (w/v) are self made.

The gel cast apparatus is assembled according to the manual (Amersham Biosciences) and filled in the designated reservoir with displacement buffer pH 8.8 (0.375 M Tris, 50% (v/v). glycerol, 0.002% (w/v) bromophenol blue).

The gel polymerisation solution pH 8.8 (12.17% (w/v) acrylamide, 0.33% (w/v) bisacrylamide, 0.375 M Tris, 0.1% (w/v) SDS, 0.05% (w/v) ammonium peroxodisulphate) is mixed in a container having a tapering nozzle and then degassed for 5 min. in the ultrasonic bath. Then, the polymerisation reaction is started by addition of 0.04% (v/v) TEMED. Immediately, the container is mounted on a stand and connected to the gel cast apparatus via a tube. The gel solution is allowed to flow into the apparatus until it stands about 3 cm under the lower edge of the gel cassettes. Then, the plug of the reservoir for the displacement buffer is engaged and the buffer displaces the gel solution until this has risen up to about 1 cm underneath the glass edge of the cassette. The cast gels are overlaid with water saturated n-butanol until complete polymerisation.

One focusing gel each is removed from the −80° C. freezer and transferred into an equilibration tube. The protein is focused in the cells and reduced by addition of 15 ml reducing buffer pH 8.8 (6 M urea, 50 mM Tris, 30% (v/v) glycerol, 4% (w/v) SDS, 65 mM DTT) each under shaking for 15 minutes at 25° C. Then, the reducing buffer is discarded and the proteins are alkylated by addition of 15 ml alkylation buffer (6 M urea, 50 mM Tris, 30% (v/v) glycerol, 4% (w/v) SDS, 260 mM iodoacetamide) with iodoacetamide. The incubation is again carried out for 15 minutes at 25° C. under shaking. Subsequently, the buffer is also discarded and the gel strip is removed from the tube. The gel is placed on the SDS gels with the aid of tweezers and is overlaid with 2 ml liquid agarose solution pH 8.3 (0.5% (w/v) agarose, 25 mM Tris, 192 mM glycine, 0.1% (w/v) SDS) and fixed thereby. The electrophoresis chamber ETTAN DALT II (Amershal Biosciences) is filled with 10 1 2D-running buffer pH 8.3 (25 mM Tris, 192 mM glycine, 0.1% (w/v) SDS), the PAA gels are inserted in the device and the electrophoresis run is performed. At this, at first a constant power of 5 W per PAA gel is set for 50 min. The temperature is constant at 20° C. Then, the power is raised to 55 W per gel, maximally, however, to 180 W, and the electrophoresis is continued until the blue control dye (bromophenol blue) has reached the lower end of the gels. The electrophoresis is stopped and the gels are removed. Per gel 400 ml (7% (v/v) acetic acid, 10% (v/v) methanol), each are placed in a tray, the gel is removed from the glass plate and transferred into the trays. For fixation the gels are incubated for 30 min at 25° C. under shaking. Meanwhile 400 ml SyproRuby staining solution are placed in a black tray and the fixed gels are transferred into the staining solution after the incubation time has elapsed. After staining for 16 h under shaking the gels are destained for 15 min in 400 ml fixation and scanned for documentation by a FLA 5000 Scanner (Fuji) at an excitation wavelength of 473 nm and an emission wavelength of 575 nm at a resolution of 100 μm and 16 bit gradiation. Then, the gels are sealed in plactic foil and stored at 4° C. until further use.

Identification of Differential Spots

For identification of differential protein spots, at first, per pH gradient (3.5-4.5; 4.0-5.0; 4.5-5.5; 5.0-6.0; 5.5-6.7; 6.0.9.0) 10 gels each with freshly prepared BMECs and 10 gels from AOECs (Aorta Endothelial Cells) were prepared and scanned. The gels were then compared using Z3-evaluation software (Compugen) and differential spots were annotated. At his, a minimal spot size of 100 pixels was assumed as a filter. The protein spots that were detected to be higher (3-fold amount or more) or unique in BMECS were cut out and transferred into a 0.2 ml reaction vessel. The cut out spots were labelled and stored at −80° C. until further use.

Hydrolysis and Mass Spectrometrical Analysis of Protein Samples

The cut out protein, which was fixed in the gel matrix, was removed from the −80° C. freezer and washed by addition of 100 μl bi-distilled water. Regarding this, the respective batch was incubated for 20 min. at 25° C. under shaking and then, the supernatant was pipetted off and discarded. This procedure was repeated for two additional times. Then, it was overlaid twice with 100 μl 50% (v/v) acetonitrile and incubated for 15 min. each at 25° C. under shaking. Again, the supernatants were discarded. The gel piece was dehydrated completely by addition of 100 μl 100% acetonitrile and 15 minutes incubation at 25° C. under shaking. After removing the supernatant the gel piece was air-dried for 5 min. Then, the gel piece was re-hydrated again in 15 μl hydrolysis buffer (50 mM (NH₄)₂CO₃), 25 ng-50 ng/15 μl trypsin V) and swollen. Hydrolysis of the proteins was carried out by incubation at 37° C. for 18 h. For the preparation of a peptide finger print via Matrix Assisted Laser Desorption Ionisation (MALDI) the hydrolysis are acidified with 15 μl 0.1% (v/v) trifluoroacetic acid. The ZipTip-C18 pipet tips used are prepared by threefold rehydration with 10 μl 50% (v/v) acetonitrile each and subsequent three-fold equilibration with 10 μl 0.1% (v/v) trifluoroacetic acid each. The application of the sample is carried out by seven up to ten-fold sucking up of the supernatant of the hydrolysis preparation. The ZipTips are then washed with 10 μl 0.1% (v/v) trifluoroacetic acid. The peptides are then eluted directly together with the matrix (α-cyano cinnamic acid, 50% (v/v) acetonitrile, 0.1% (v/v) trifluoroacetic acid) by three up to four-fold pipetting up and down on the MALDI-measure carrier. After drying the samples on the carrier, the samples are at first measures mass spectrometrically via MALDI fingerprint and analysed. For this, the samples are measured at the Voyager DE PRO (Per-Spective Biosystems) MALDI-mass spectrometer in positive reflector mode with an acceleration voltage of 20,000 V, a grid voltage of 75%, a guide wire of 0.02% and a delay time of 220 ns. At his, a mass window is used for masses between 700 and 3500 Da.

The mass lists obtained for each protein spot are employed in a database query. At his, three different programmes are used: Mascot, MSFit and Profound.

Protein spots, for which a database identification is not possible despite a good mass fingerprint are used in ESI-mass spectrometry for the generation of amino acid sequence information.

For this, after hydrolysis the hydrolysis preparation is acidified with 15 μl 0.2% (v/v) formic acid and incubated for 30 min under shaking. ZipTip-C18-pipette tips (MilliPore) are meanwhile re-hydrated threefold with 10 μl 50% (v/v) acetonitrile each and subsequently equilibrated by threefold washing with 10 μl 0.1% (v/v) trifluoroacetic acid each. The application of the sample is carried out by seven up to tenfold sucking up of the supernatant of the hydrolysis preparation. The ZipTips are then washed with 10 μl 0.1% (v/v) trifluoroacetic acid. Subsequently, the buffer is exchanged. by two-fold washing with 0.1% (v/v) formic acid and the peptides are then eluted by five up to seven-fold sucking up of 2 μl 50% (v/v) ethanol.

The peptide mixtures obtained can either be analysed directly or analysed via liquid chromatography (LC) coupled with ESI-mass spectrometry.

For a direct measurement 1 μl of the eluted samples is filled in a hollow needle (Protana). At this, at first a overview spectrum with an ion spray voltage of 850-1,000 V, a curtain gas pressure of 20 psi, a declustering potential of 40-50 V, a focusing potential of 245 V and a voltage at the multi-channel plate of 2,000-2,100 V is measured in positive mode. In doing so, the scanning area is at 100-1,600 or 410-1,600 Th. The peptides detected in the spectrum are subjected to a collision induced fragmentation.

For that purpose, product ion spectra of each peptide are recorded at an ion spray voltage of 850-1,000 V, a curtain gas pressure of 20-40 psi, a declustering potential of 40-50 V, a focusing potential of 245 V, a quadrupole resolution of 0.7-1.0 amu, a collision energy of 15-50 V and a voltage at the multi-channel plate of 2,100-2,400 V. At this, the scanning range 50-1,600 Th.

Upon coupling the nanoHPLC to the ESI mass spectrometer, at first, the reversed phase (RP) pre-column is loaded with 2 μl sample at a flow of 20 μl/min. 0.1% (v/v) trifluoroacetic acid. The chromatographic separation of the peptides is carried out via a RP-C18 column (LC packings) with the gradient over 35 min. from the starting conditions (0.05% (v/v) formic acid, 10% (v/v) acetonitrile) to the final conditions (0.05% (v/v) formic acid, 76% (v/v) acetonitrile) . Coupling of the HPLC with the mass spectrometer is carried out via a hollow needle (New Objective). The settings of the mass spectrometer are selected such that during the LC-run two experiments can be performed. The parameters set at this, except the ion spray voltage (1,800-2,200 V), correspond to the once already set forth above. Both overview spectra and product ion spectra are recorded alternately during the run. The settings for the product ion spectra are selected such that the two most intensive signals of the overview spectra, which are charged two-fold, three-fold or four-fold and the intensity of which is greater than 10 cps, are subsequently analyzed via a collision-induced fragmentation. At this, the scanning range is from 450-1,600 Th. The evaluation of the spectra obtained is carried out in three steps:

A) The product ion spectra containing information about the amino acid sequence of the corresponding peptide are first compared completely with public databases with the aid of the software programme MASCOT (Matrix Sciences). If, in doing so, the peptide cannot be matched to a protein,

B) at first, the product ion spectra are automatically sequenced with a software tool of the manufacturer of the device. The such obtained amino acid sequences were compared via MSBlast with public databases according to Shevchenko et al. If the protein could not be identified,

C) the product ion spectra were evaluated manually and were the amino acid sequences obtained compared with public data-bases via blast or FASTA.

According to the method described above, BBB-specific proteins or also fragments thereof can selectively be identified in endothelial cells of brain capillaries. The preceding description of course allows routine variations, which are obvious to a person skilled in the art. For Example, the following processing steps can be varied:
As digest methods also other methods for obtaining the proteins known to the person skilled in the art can be used, which are described in standard literature, (e.g. “2D Proteome Analysis Protocols”)
For the isoelectric focusing respective focusing gels from other manufacturers can of course be employed. Also different length and pH gradients can be employed.
For the separation in the second dimension respective gel systems of other manufacturers can of course be employed. The use of further gel sizes is possible, too.
According to the standards also other proteases known to a person skilled in the art can be used for preparing the peptide pattern.
Mass spectrometers of other types and other manufacturers can also be used for the determination of peptide masses and of the de novo amino acid sequences.
The mass spectrometric conditions can be varied both apparatively and functionally corresponding to the sample for the determination of peptide masses and for the de novo amino acid sequencing.
   Western Blot of the Proteins

At first, proteins were separated on 12.5% polyacrylamide gels as described above and subsequently transferred onto nitrocellulose membranes.

At this, seven Whatman papers (Schleicher & Schüll) were cut corresponding to the size of the separating gel and each soaked with different buffers.

Two papers in anode buffer I (300 mM Tris base, 20% (v/v) methanol) were placed without air bubbles onto the anodes of the blooding apparatus (BioRad), followed by two papers in anode buffer II (25 mM tris base, 20% (v/v) methanol) . The nitro cellulose membrane, which was also soaked in anode buffer II, was placed thereon, then the polyacrylamide gel followed. Finally, three further papers were soaked in cathode buffer (25 mM Tris, 40 mM amino caproic acid, 0.1% (w/v) SDS, 20% methanol) and applied. The apparatus was closed and the transfer of the proteins occurred for one hour at maximum 25 V and 2.5 mA/cm² gel.

Then an immunochemical staining of the proteins was carried out with polyclonal antisera from rabbits.

Regarding this, the membranes were washed in TBST buffer (10 mM Tris base, 150 mM sodium chloride, 0.05% (v/v) Tween 20; pH 8.0) and, subsequently, free binding sites were saturated with Blotto (10 mM Tris base, 150 mM sodium chloride, 0.05% (v/v) Tween 20, 5% (w/v) skim milk powder; pH 8.0). The incubation with the first antibody was carried out for two h at RT in TBST buffer [anti-EMP1-antibody (rabbit) 1:4000; anti-TKA-1-antibody (rabbit) 1:4000], then it was washed three times with TBST buffer. The detection of bound antibodies occurred via incubation with a secondary antibody conjugated to alkaline phosphatase for 1 h at RT in TBST buffer [anti-rabbit-IgG-antibody (goat) 1:5000]. After two-fold washing with TBST buffer, the membrane was re-buffered to an alkaline pH value by incubation with AP buffer (100 mM Tris base, 100 mM sodium chloride, 5 mM magnesium chloride; pH 9.5). As the substrate for the colour reaction 0.016% (w/v) nitrotetrazolium blue chloride and 0.033% (w/v) 5-bromo-4-chloro-3-indolylphosphate-disodium salt in AP-buffer were used.

In the following, the identification of BBB-specific proteins or fragments thereof in endothelial cells of brain capillaries via the genomics approach will be described.

Identification of BBB-Specific Transcripts via cDNA Subtraction

The selective identification of cell or tissue specific proteins is carried out via a differential method. This can be carried out on protein level via the comparison of 2D gels of digests of various tissues and cells, respectively, and by subsequent determination of the proteins specific for a tissue and a cell type, respectively. In order to be independent of the physical features of proteins (size, solubility) also differential methods on the level of transcription can be carried out for identifying specific proteins. Such subtractive RNA techniques additionally have the advantage of requiring less tissue and cell material, respectively.

For the identification of BBB-specific proteins the use of freshly isolated BMEC as starting material is crucial. Methods described up to now at the best were based on the subtraction of RNA from brain capillaries against RNA from kidney (Li et al., 2001). At this, it is problematic that brain capillaries next to BMEC also contain other cell types such as pericytes and astrocytes. Moreover, kidney as a subtraction tissue is very heterogeneous since it consists of different cell types of which endothelial cells only comprise a small portion. According to the invention, a subtraction tissue is to be used that enables a selective identification of transcripts and proteins, respectively, specific for the blood-brain barrier. Basically, any endothelial cells can be used as comparative tissue, for example, macro- and microvascular endothelial cells of the same tissue or also endothelial cells from other organs, e.g., heart, lungs, kidney, liver, aorta, etc. can be used as comparative tissue. Also de-differentiated BMEC attained from culture can be used. However, it is preferable to use another endothelial cell type as comparative tissue vis-à-vis endothelial cells of brain capillaries. Preferably used are endothelial cells from aorta, which exhibit no barrier function. This, additional has the advantage that microvessels can be compared with macro vessels. Furthermore, also other micro vascular endothelial cells can be used. Also suitable as comparative tissue are endothelial cells of brain capillaries cultivated under other conditions, e.g. under other conditions as regard pH value, growth matrix, growth factor such as cytokines. The physiological significance of the identified targets follows from the known features of endothelial cells of brain capillaries vis-à-vis the respective comparative tissue. Two defined cell types are preferably used according to the invention: Freshly isolated BMEC as the cell type with barrier function and endothelial cells from aorta, which like BMEC are also endothelial cells, yet without exhibiting barrier function. This approach allows to identify transcripts and proteins, respectively, contributing to the formation of the blood-brain barrier, much more selectively.

Preparation of the Subtractive cDHA Library

Total RNA is isolated from the cells using Trizol (Invitrogen) according to the manufacture's specifications. The total RNA is subsequently checked on a denaturating agarose gel for its integrity. For RNA isolation 100 mg tissue and 10 cm² confluently grown cells, respectively, in 1 ml Trizol each are homogenized mechanically and the homogenate is subsequently incubated for 5 min. at RT. Thereafter, 0.2 ml chloroform/1 ml Trizol (Invitrogen) are added, mixed by Vortex for 15 sec. and incubated at RT for 3 min. For phase separation it is centrifuged for 15 min. at 4° C. and 12,000×g. Following that, the upper aqueous phase is transferred in a fresh container. 0.5 ml isopropanol/1 ml Trizol are added followed by mixing and incubation at RT for 10 min. The RNA is sedimented by centrifugation for 10 min. at 4° C. and 12,000×g, washed twice with 75% EtOH, air-dried and dissolved in DEPC-treated water. The concentration is determined spectro-photometrically and the quality is checked in a denaturing agarose gel.

Starting from total RNA the mRNA is enriched by using Dyna-beads (Dynal) according to the manufacturer's specifications.

mRNA enrichment: 75 μg total RNA are denatured for 2 min. at 65° C., immediately added to 200 μl Dynabeads Oligo (dT)₂₅ (Dynal) in two-fold binding buffer and incubated for 5 min. under mixing. The supernatant of the magnetic separation is discarded and the Dynabeadas are washed twice with washing buffer. The polyA⁺-RNA is finally eluted with 20 μl 10 mM Tris-HCl pH 7.5 for 2 min. at 85° C.

The preparation of the subtractive cDNA library can be carried out with commercial PCR subtraction kits, for example, the PCR-Select cDNA subtraction kit of the company Clontech can be used according to the manufacturer's specifications.

For this, 2 μg mRNA from BMEC (tester) and AOEC (driver) each are transcribed into single stranded cDNA with the enzyme AMV Reverse Transcriptase, starting from an oligo(dT) adapter primer directly following that the synthesis of the second strand is carried out with an enzyme mix (DNA Polymerase I, RNase H and DNA ligase) for two hours at 16° C. and with subsequent addition of T4 DNA polymerase and further incubation at 16° C. for 30 minutes. The such prepared double-stranded cDNA is purified via phenol/chloroform extraction and ethanol precipitation.

For the introduction of suitable ends for the later adapter ligation as well as for the generation of a more uniform size: distribution of the cDNA fragments, a restriction with Rsa I is now carried out. The in this way prepared double-stranded cDNA fragments are purified via phenol/chloroform extraction and ethanol precipitation. The products of the cDNA syntheses as well as the restrictions are gelelectrophoretically checked for purity.

For later amplification via PCR the adapters 1 and 2R are now added with the enzyme T4 DNA ligase to the tester cDNA via the Rsa I ends. The ligation is checked via PCR.

The actual subtraction takes place by means of two hybridisations. For the first hybridisation in one batch cDNA for BMEC adaptor 1 is hybridized with AOEC cDNA, in another batch cDNA from BMEC adapter 2R with AOEC cDNA. In the second hybridization both batches from the first hybridisation are combined and hybridized with freshly-denatured cDNA from AOEC.

The products of the hybridization are finally used as template in a first PCR reaction; an oligonucleotide from the common region of both adapters 1 and 2R serves as a primer.

The product mixture of this first PCR was now used as template in a nested PCR, wherein the two primers arranged within each other are each derived from the unique region of both adapters 1 and 2R. This second PCR increases the specificity.

The efficacy of the subtraction was checked through comparative PCR for a housekeeping gene (GAPDH): With the cDNA from the subtraction a product formation can take place only after significantly more PCR cycles when compared to the two non-subtracted cDNAs from BMEC and AOEC. GAPDH as a typical housekeeping gene is expressed in all tissues and cell types to a comparable extent. Therefore, upon subtractive hybridization, it should not be enriched as differentially expressed genes but the amount of transcript should significantly be decreased in the subtracted cDNAs (both forward and reverse subtraction) compared to the cDNAs from BMEC and AOEC, respectively, before subtraction. This is experimentally confirmed in that both cDNAs before subtraction and the respective subtracted cDNAs, respectively, are used for a PCR with GAPDH-specific primers. Since with the subtracted cDNAs a first product formation is only obtained after additional 16 cycles as compared to the two not-subtracted cDNAs and enrichment of at least a factor of 50,000 consequently occurs through the hybridization. This enrichment facilitates the selective identification of BBB-specific transcripts and already represents a first validation of the isolated sequences as well.

The products of the second PCR are cloned in the vector pT-Adv (Clontech) and transformed into TOP10F′ (Clontech) chemocompetent E. coli. The products of the second PCR are cloned in the plasmid vector pT-Adv (Clontech). This vector has overlapping dT residues at the 5′ ends, which are compatible to the 3′ dA residues, that are e.g. attached via Taq DNA polymerase to PCR products. This and comparative, respectively systems allow the direct cloning of PCR products with high efficacy. The transformation is carried out in chemocompetent E. coli TOP10F′ (Clontech) as described in the literature (Sambrook et al., 1989).

Differential Hybridisation

Clones from the subtractive cDNA library are verified by differential hybridization as regards their expression BMEC vs. AOEC. Regarding this, the PCR-Select Differential Screening Kit (Clontech) is used. The reverse subtracted probe was prepared with the PCR-Select cDNA Subtraction Kit of the company Clontech according to the manufacturer's specifications described above wherein BMEC serves as driver and AOEC as tester.

According to the manufacturer's specifications, liquid cultures of the clones are inoculated in 96 well microtiter plates. These are employed as templates for the amplification of insertions with the primers adapter 1 and 2R. The remainder of the liquid cultures is added with glycerol and frozen as permanent culture. The PCR products are checked gelelectrophoretically. 1 μl each of products which were bigger than 200 bp were spotted on two identical HybondN membranes and fixed thereon using UV-light. Deviating from the manufacturer's specifications only two filters having 92 clones are hybridized each time: a filter with the forward subtracted probe, in which the BMEC-specific transcripts are enriched and the other filter with the reverse subtracted probe in which the AOEC-specific transcripts are enriched. The hybridization of two further filters with cDNA from BMEC and AOEC, respectively, was omitted, since no relevant additional information can be gathered therefrom. Instead, RNA from BMEC and AOEC is used for preparing expression patterns at the later verification in Northern blot analysis and RT-PCR experiments, respectively. 92 clones from the subtractive cDNA library as well as two negative controls of the manufacturer are applied per filter. Additionally to the manufacturer's specifications one PCR product of a housekeeping gene being equally strong expressed in BMEC and AOEC is spotted as well as a PCR product for a BHS marker (Apolipoprotein Al), which is stronger expressed in BMEC than in AOEC, as a positive control.

The hybridizations are performed with probes of equal activity at 72° C. with ExpressHyb solution (Clontech) as described by the manufacturer. Subsequently, the filters are washed stringently. The common conditions of stringency can be used. Favourably, the filters are washed for 2×20 min at 68° C. up to a stringency of 0.2×SSC/0.5% SDS. The signal intensities are determined via expositions of different duration on a film by means of a phosphoimager (FLA-5000, Fuji). Clones showing an about five-fold stronger signal in BMEC than in AOEC are classified as differentially expressed and are processed further.

Liquid cultures from the permanent culture of positive clones are inoculated and the plasmid DNA is isolated according to standard methods (Birnboim and Doly, 1979) by means of Quiagen-columns. The insertions of plasmids are sequenced with universal primers and optionally with additional gene-specific primers. With the DNA sequences obtained databases are searched for homologies by using the algorithms BLAST (http://www.ncbi.nlm.nih.gov/BLAST) and FASTA (http://www.ebi.ac.uk/fasta33).

Further Verification of BBB-Specific Transcripts: Expression Pattern

From the positive clones of interest expression patterns are generated in BMEC, AOEC and nine further tissues. This is carried out via RT-PCT and/or Northern blot analyses.

For RT-PCR experiments cDNAs are prepared by random priming, starting from total RNA. All enzymes used as well as random hexameres are from Invitrogen. For this 5 μl DNase I 10× buffer as well as 5 μml DNase I are added to 10 μg total RNA each in 40 μl nuclease-free water and incubated for 15 minutes at 25° C. Subsequently, 5 μl 25 mM EDTA are added and the enzyme is heat-inactivated for 15 minutes at 65° C. 25 μl are removed from that preparation, filled up to 100 μl with nuclease free water and stored as —RT control at −80° C. 8 μl random primer (100 ng/μl), 3 μl dNTP-mix (10 mM each) and 2 μl nuclease-free water are added to the remaining 25 μl total RNA from the DNase I digest. Now RNA secondary structures are disruped for 5 minutes at 65° C. and the sample is put on ice immediately thereafter. 10 μl 5× first strand buffer, 6 μl DTT (100 mM) and 3 μl RNaseOUT are added, it is incubated for 10 minutes at 25° C. for primer annealing and subsequently the temperature is adjusted to 42° C. for two minutes. 3 μl Super-Script II Reverse Transcriptase are added and is incubated at 42° C. for 50 minutes. Then the enzyme is heat-inactivated for 15 minutes at 70° C. 3 μl RNase H are added and it is incubated for 20 minutes at 37° C. in order to degrade the total RNA from the cDNA. Finally, it is filled up to 100 μl with nuclease-free water and the cDNA is stored at 80° C. The quality of the cDNAs is checked via PCR with primers for a house-keeping gene (GAPDH) and for 18S rRNA, respectively. At this it is to be expected that comparable amounts of products are generated in each case with cDNAs from different tissues and cells, respectively. The prepared cDNA cells are in each case used for preparing expression patterns for the transcripts to be investigated.

For Northern blot analysis for preparing expression patterns, the total RNA from the cells and tissues, respectively, as separated in denaturing gels according to its size, transferred onto a nylon membrane. There, it is hybridized with radioactively labelled gene specific probes. 6.0 g agarose is dissolved in 290 ml DEPC-treated water under heating. Then it is cooled to 60° C. in a water bath on 60° C. and 40 ml 10× MOPS-buffer (200 mM MOPS, 50 mM sodium acetate, 10 mM EDTA) as well as 70 ml formaldehyde are added. Finally, a maxigel with a big pocket former (12 lines) is cast in the fume hood and left there-to solidify. 15 μg total RNA each in 10 μl are denatured with 40 μl sample buffer (500 μl deionised formamide, 160 μl formaldehyde, 100 μl 10× MOPS, 240 μl DEPC-treated water) for 15 minutes at 65° C. and subsequently transferred onto ice. Now 10 μl loading buffer (500 μl glycerol, 2 μl 500 mM EDTA, 25 μl 10% bromophenol blue, 473 μl DEPC-treated water) are added and the sample is applied on the gel which is overlaid with 1× MOPS buffer. The electrophoresis is performed for 3-4 h at 250 V. Thereafter, the gel is first swivelled in water for 10 min, subsequently in 10×SSC for 30 minutes. A Hybond XL filter tailored to fit the gel size is shaken for 15 minutes in 10×SSC.

Assembly of the blot (from bottom to above): salt bridge (immerses in buffer reservoir with 10×SSC), gel, filter, 5 3 MM (previously immersed in 10×SSC), about 7 cm green towels (cellulose cloths), glass plate, weight of about 0.5 kg. The blotting takes place for 16-20 h. Then the blot is disassembled and the Hybond XL filter is washed for 10 minutes in 2×SSC. The RNA is now fixed onto the Hybond filter in a UV-crosslinker with 70,000 μJ/cm². Then the filter is stained for 1 minute in staining solution (300 mg methylene blue in 1 L 0.3 M sodium acetate) in order to visualize the RNA, and then washed with water for 2 minutes to destain the back-ground. The stained filters are photographically documented. Subsequently, the filter is dried between 3 MM paper, wrapped in saran wrap and stored at −20° C.

The hybridizations are carried out with radioactively labelled cDNA probes (Rediprime II, Amersham) which have been purified over ProbeQuant G-50 columns (Amersham) by using ExpressHyb solution (Clontech) according to the manufacture's specifications. After a first checking of the hybridization by means of a phosphoimager, FLA-5000 (Fuji) autoradiograms are prepared on Biomax MS films (Kodak).

Completion of cDNA Sequences

The complete cDNA sequences for BBB-specific clones from the subtractive cDNA library of interest are determined by screening various cDNA libraries and RACE-PCR experiments.

A cDNA library is built from BMEC from pig with the SMART cDNA Library Construction Kit (Clontech) in the vector λTriplEx2 according to the manufacturer's specifications. For this, at first, total RNA is isolated with Trizol (Invitrogen) as described above and polyA⁺-RNA is enriched therefrom with aid of Dynabeads (Dynal). For the preparation of the library 2 μg PolyA⁺-RNA from BMEC are employed. Finally, the ligations are packaged in vitro with the phage extract Gigapack III Gold (Stratagene) according to the manufacturer's specifications. The number of independent phages of the cDNA library from BMEC amounts to 1.3 million pfu, from which more than 99% were recombinant upon performing the blue/white test (cf. Sambrook et al., 1989). At least half of the inserts had a size of more than 1 kb. After amplification of the complete library the titer is about 2×10¹⁰ pfu/ml at a total volume of about 150 ml. This page lysate is adjusted to 7 (v/v) % DMSO and stored at −80° C. The phage library described is converted into a plasmid library according to the manufacturer's specifications (Clontech ClonCapture cDNA Selection Kit) in that E. coli BM25.8 are infected with 2 million pfu of the phage library. This bacterial strain expresses Crerekombinase which recognizes the loxP-sites in the vector λTriplEx2 and thus enables the convertion. At this, the convertion of lamda phages in the plasmids takes place via in vivo excision and subsequent circularisation of the complete plasmid. The plasmids obtained are then stably passed on an E. coli. The plasmid preparation is carried out from plate cultures of infected BM25.8 with the NucleoBond Plasmid Kit (Clontech).

For screening cDNA plasmid libraries with ClonCapture biotinylated cDNA probes are employed. These form DNA triplex structures with homologous sequences of the plasmid insertions in a RecA mediated reaction. The thus selected plasmids can be isolated via streptavidin coupled to magnetic beads and employed in a transformation. Clones from such enrichment are then screened by colony hybridization, plasmid DNA is isolated and sequenced from positive clones resulting there-from.

The isolation of positive clones by ClonCapture is performed exactly according to the manufacturer's specifications (Clontech). For the preparation of the probe, at first, a PCR with gene specific primers was optimized for a suitable plasmid so that only one product formed. For this, the primers are designed such that they have melting temperatures that differ no more than 1° C. from each other and neither form primer dimmers nor stabile loops. The annealing temperature is selected relatively high with being 2-5° C. under the melting temperature calculated according to the formula Tₘ=[(G+C)×4]+[(A +T)×2]. This leads to specific product formation what results in only one band at the control via gelelectrophoresis. From this product, a piece is removed from an agarose gel with a sterile Pasteur pipette and transferred into 200 μl sterile water. By vortexing and incubating for 30 minutes at 70° C., the DNA is eluted from the gel piece and serves as template for the preparation of the probe. With this template control reactions are performed with and without biotin-21-dUTP and analyzed in an agarose gel, since biotin can inhibit that PCR. Upon successful control reaction the biotinylated probe is now prepared in a preparative PCR addition of 10 μCi [α³²P]dCTP. After 20 cycles 5 μl from the preparation are checked on an agarose gel and 5 further cycles are possibly added. Subsequently, the PCR product is purified with the NucleoSpin Extraction Kit (Clontech) according to the manufacturer's specifications and eluted with 35 μl elution buffer. From that 2 μl are analyzed gelelectrophoretically and the product is quantified spectrophotometrically. For controlling the biotinylation 2 μl of the purified PCR: product are added to 15 μl magnetic beads and the preincubation signal is determined with a Geiger counter. After 30 minutes of incubation under slight shaking the magnetic beads are separated off in the magnet and the supernatant is quantified again with the Geiger counter (post-incubation signal). Upon successful biotinylation the pre-incubation signal is 2-4-fold stronger than the post-incubation signal.

For the capturing 50 (200 bp)−100 (600 bp) ng biotinylated PCR product in water are denatured for five minutes at 100° C. and is then immediately transferred onto ice. Now all components except the plasmid-DNA are added wherein 2 μg RecA protein per 50 ng probe are employed. After 15 minutes of incubation at 37° C. 1 μg of the plasmid library is added and it is incubated for additional 20 minutes at 37° C. In the meantime 15 μl magnetic beads are unspecifically saturated with herring sperm DNA and prepared for the purification of the capturing. EcoR V cleaved λ-DNA is added to the capturing and a proteinase K digestion is carried out for 10 minutes at 37° C. This reaction is finally stopped by addition of PMSF and the capturing preparation is purified via the magnetic beads. The isolated plasmids are eluted with 100 μl elution buffer, precipitated and subsequently dissolved in 10 μl water.

2 μl of the plasmid library enriched by the ClonCapture are transformed in electrocompetent E. coli DH5α and plated out of LB-Amp plates. Positive clones are identified by colony hybridization with radioactively marked probes (same amplicon as with biotinylation) according to standard methods. The thus obtained clones are further verified by colony PCR for which a primer from the mentioned amplicon and another primer which is located downstream, is used. It is to be avoided to chose both primers from the amplicon that has been used as probe for ClonCapture in order to avoid during colony PCR [PCR is performed in which bacteria from an individual colony are used instead of DNA] that product formation does not occur at the plasmids contained in the bacteria but due to contaminating probe. Therefore, at least 1 primer should be located outside the amplicon in the best case 3′ to it, because this sequence is both known and contained in all positive clones of the cDNA library. The plasmid DNA of positive clones was isolated according to standard methods (Birnboim and Doly, 1979) with the aid of Qiagen columns and sequenced according to the chain termination method (Sanger et al., 1977) For sequencing the “ABI Prism BigDye Termiantor Cycle Sequencing Ready Reaction Kit, Version 2.0” (Applied Biosystms) can be used according to the manufacturer's specifications. The products of the sequencing reactions are analyzed upon the “ABI Prism 310 Genetic Analyzer” (Applied Biosystems).

The RACE-PCR (Frohman et al., 1988) serves for determining unknown cDNA sequences, starting from a known sequence section by cDNA synthesis followed by the introduction of known synthetic ends for annealing the second PCR primers.

The 5′RACE-PCR is performed with the 5′ RACE System for Rapid Amplification of cDNA Ends, Version 2.0 (Invitrogen) according to the manufacturer's specifications. At this, first a cDNA primary strand synthesis takes places with a gene specific primer (GSP1) and 1 μg total RNA from BMEC. After purification of the cDNA over GlassMAX-columns, in a second step, an oligo-dC-tail is attached with the aid of the enzyme terminal desoxynucleotide transferase. The first PCR takes place on 5 μl tailed cDNA with a further gene-specific primer (GSP2) and the abridged anchor primer, which attached to the oligo-dC-tail. The specificity of the PCR was increased by means of a second nested PCR which is performed with the abridged universal amplification primer and a third gene-specific primer (GSP3) on 5 μl 1:100-diluted PCR product from the first PCR. Batches with only one primer each as well as a water-control serve as controls at the second PCR. After gelelectrophoretic analysis the product of the second PCR is possibly cloned for which a ligation with the pGEM-Teasy System II (Promega) and transformation into electrocompetent DH5α are performed. The clones obtained are examined by means of colony PCR, the plasmid DNA is prepared and finally sequenced in actually known manner.

The 3′RACE-PCR can be performed with the 3′RACE System for Rapid Amplification of cDNA Ends (Intvitrogen) according to the manufacturer's specifications. At the 3′RACE-PCR the cDNA primary strand synthesis is carried out for 5 μg total RNA from the BMEC with the oligo-dT adapter primer. For the first PCR 2 μl cDNA with a gene specific primer (GSP1) and the abridged universal amplification primer are employed. A seminested second PCR is performed with the gene-specific primer (GSP2) and the abridged universal amplification primer together with the controls as described for the 5′RACE. The products are cloned and sequenced as described.

According to the above-described method BBB-specific proteins or also fragments thereof can be selectively identified in endothelial cells of brain capillaries. The above description of course allows routine variations which are obvious to a person skilled in the art. For example, the following processing steps can be varied:
Isolated-BMEC can be disseminated and a primary culture can be used as tester at the subtraction instead of fresh BMECs.
Another subtractive tissue (driver) e.g., dedifferentiated BMEC from the culture (at least passage 2) can be chosen.
RNA and mRNA, respectively, can be prepared according to any other method known to a person skilled in the art under the proviso that the RNA is intact and the mRNA can be transcribed into cDNA with reverse transcription, respectively.
The PCR products from the subtraction can be cloned in each suitable vector system, both via polymerase-caused 3′dA residues and via blunt ends or after restriction.

The transformation can take place into different E. coli strains both into chemically and electrocompetent cells as it is well known in the art.
The step of the differential hybridization is optional but recommendable. At this, also other suitable membranes (e.g., positively charged or uncharged nylon membranes) as well as other hybridization solutions can be used. Stringent washing of the membranes can also be achieved at other temperatures and with other solutions, respectively, (e.g. lower temperatures and lower salt content, and higher temperature and higher salt content, respectively, e.g. T=50-70° C., 0.5-0.05×SSC/O.1% 5DS).
Expression patterns can also be determined via quantitative PCR (real time PCR) with the respective cDNAs. Practically, the quantitative PCR is performed with the opticon (MJ Research). For performing the reaction the “QuantiText SYBR Green PCR Kit” of Qiagen is used, wherein PCR conditions as described above are used. For quantification one dilution series each is prepared from BMEC cDNA. The specifications are carried out in picograms of used RNA equivalents. For performing the relative quantification, in each case, the calculated amounts of target are divided by the calculated amounts of 18S rRNA. Finally, a sample, e.g., BMEC, is set as 100% and all other samples are referred thereto.
The cDNAs can also be produced with the aid of other systems. Northern blot analyses can also be performed with other suitable probes and hybridization/washing solutions.
cDNAs can also be extended via database mining by means of known overlapping sequences. Experimentally, also any other cDNA libraries from the cells and tissues, respectively, in which the transcript sought after occurs can be screened with various systems. RNA from cells and tissues, respectively, in which the transcripts sought after occurs, can be employed in the RACE-PCR, respectively, (cf. Sambrock, 1989). For RACE-PCRs any other suitable systems known to a person skilled in the art can be used.

The proteins or fragments identified with this method have a specificity for the blood-brain barrier and are subject matter of the present invention, as well. The knowledge of the specificity of a protein or fragment thereof for the blood-brain barrier now allows the selective discovering of the function of the protein. Normally, the determination of the function is carried out via comparison with known sequence data in available databases, for example, by using the BLAST algorithm. Knowledge of the specificity of the identified proteins further allows a selective modulation of the expression in the blood-brain barrier whereby pathological conditions can specifically be treated.

That way, agonists or antagonists for the respective BBB-specific proteins can be developed which selectively modulate their activity. The expression of such proteins can also be modulated directly, e.g., via gene transfer or antisense RNA. Particularly, attractive for therapeutical approaches is the development of “Trojan Horses”-medicaments, which are coupled to molecules that are actively transported across the BBB by identified transporters. Also so-called prodrugs, substances which are modified by BBB-specific enzymes in the endothelial cells and thus obtain their therapeutical effect, are possible.

BBB-specific proteins fulfil manifold functions. For example, they serve for the supply of nutrients (example glucose transporter GLUT1) or serve as contact proteins (e.g. ZO-1 as tight junction protein). Further, they possess enzymatic activity (e.g. glutamyl transpeptidase GGT) or function as transport vehicles for amino acids.

Expression of BBB-specific proteins upon ischemia

The expression behaviour of BBB-specific proteins identified according to the invention was investigated upon ischemia. For this the endothelial cells prepared were resuspended after the washing and disseminated in cell culture bottles coated with collagen as described by Franke et. al (2000). Cultivation of the cells took place at 37° C. in CO₂ incubators having a constant CO₂ content of 5%. After the cells had reached confluence they were detached by treatment with trypsin solution and were splitted in transwell dishes (44 cm², Corning) prepared therefore. After 3 days of cultivation of the cells under the already described conditions, the transwell batch was transferred into a dish on the bottom of which C6-glioma cells (customary, e.g., purchasable from the ATCC) had been grown. The two cell types were further cultivated for two days in co-culture under the addition of hydro cortisone. An exchange of medium was undertaken for the experiment for the expression under ischemia. Before hand, the new medium was fumigated with 0.2% O₂, 94.2% N₂ and 5% CO₂ and did not contain glucose. Subsequently, the cells were stored for 24 h at 37° C. in CO₂ incubators with 0.2% O₂, 94.2% N₂ and 5% CO₂. An exchange of medium was also performed for the control. Before hand, the medium was fumigated with 21% O₂, 74% N₂ and 5% CO₂ and contained glucose. The cells were cultivated further for 24 h under these conditions. Then the expression of the respective protein was determined quantitatively as described above.

Now, the following proteins at the blood-brain barrier were identified with the method of the invention.

### EXAMPLE 1

### Identification of S129=ITM2A

BMEC that were freshly isolated from the brain of pigs as described above and that were purified or cultivated in M199 media (Sigma) with 10 (v/v) % oxen serum (PAA) on collagen G (Biochrom) and passaged by trypsination. From cultivated BMEC total RNA was isolated as described above from the primary culture (P0) as well as from the passages 1-3 (P1-3) from a T75-cell culture bottle each. cDNA was prepared therefrom as already described and was examined as to its quality. Expression patterns comparing between fresh BMEC and P0-3 were prepared with the respective gene-specific primers, in each case with regard to GAPDH and 18S rRNA, respectively. The clones described in this example and in the following examples were obtained.

The subtractive clone S129 showed a>5-fold stronger signal as compared to the reverse probe in the differential screen with a forward probe and was, therefore, chosen for a sequencing. The sequence of clone S129 is indicated as SEQ ID NO: 1. Based on this sequence S129 was unambiguously identified as Itm2A.

At first, an expression pattern for Itm2A was prepared with the primers Itm2a.s2 (5′ ACC TCC ATT GTT ATG CCT CCT A 3′=SEQ ID NO: 2) and Itm2a.as2 (5′ GFF GCC TCT CAC TCT TGA CAG A 3′=SEQ ID NO: 3) as described above, GAPDH was used as a control. The expression pattern was obtained via RT-PCR (not depicted).

The semi-quantitative expression pattern shows that Itm2A is more strongly expressed in BMEC than is in AOEC and, thus, confirms the results of a differential hybridization. Moreover, the expression BMEC is also clearly stronger than in Cortex (brain), being an indication towards the specificity for BMEC in the brain. Merely in the heart a strong expression can be seen, which can possibly be correlated with the described expression in muscle. The expression pattern was verified by Northern blot analysis. At his, the coding region of Itm2A from pig (FIG. 1a) served as a probe.

In the Northern blot the specificity for BMEC is even more explicit. This expression in BMEC and therefore at the BBB is hitherto not described.

Further, a second, smaller transcript in BMEC can be recognized in the Northern blot. In order to characterize this, the coding region as well as the 5′ and 3′ non-coding region was investigated with RT-PCR and RACE-PCR, respectively, in BMEC. At this, it turned out that two 3′ non-coding regions exist for Itm2A, the shorter of which is created by an alternative polyadenylation signal as became apparent by sequencing. This has neither been described for Itm2A up to now. Probably, the frequency of transcription, and, therefore, also the amount of protein, is regulated by two different 3′ regions via different stabilities. The experiments described also delivered the complete cDNA sequence (complete CDS 119-910) for Itm2A from pig (SEQ ID NO: 4+SEQ ID NO: 5).

The expression of the target Itm2A/S19 under ischemic conditions was examined according to the common experimental instruction set forth above. At this, it became apparent that the target S129 in BMEC is strongly reduced in expression under ischemia. This speaks for an involvement of Itm2A in diseases connected with ischemic conditions such as stroke, cardiac infarction and tumor associated conditions, such as for example occur with a glioblastoma. The expression pattern determined for once support the usability of the target as a diagnostic marker for these diseases and for the other supports the therapeutic usability of the target for the causative treatment of the diseases mentioned above. The expression pattern of Itm2A in BMEC under ischemia when compared with a control set to 100% is shown in FIG. 1b. BMEC were cultivated once under ischemia conditions (“ischemia”) and once under normal conditions (“control”) as described in the method section. Subsequently, the expression of targets in both samples was measured relative to 18S rRNA. The value obtained was set to 100% for control and the ischemia sample was referred thereto.

In order to obtain hints for a possible role of a Itm2A at the BBB, the expression pattern in cultivated BMEC was investigated in comparison to known BBB markers and GAPDH, respectively. The result is depicted in FIG. 2. These data show a quick decrease of the expression before Itm2A as it is also described for known BBB markers. A housekeeping gene such as GAPDH, however, shows no regulation.

The data clearly points towards the function of Itm2A at the BBB. When considering the role of the protein during the differentiation in chondrocytes and T-cells one can conclude that Itm2A is also responsible for the special differentiation state of endothelial cells at the BBB. Since Itm2A is demonstrately located in the plasma membrane, in certain states of cells, here it seemingly represents a receptor by possibly forming homo- or heteromultimers. The extracellular portion of such a receptor would bind to secreted molecules or surface molecules of other cells, the intracellular portion of the receptor complex could transducer signals in such a model—e.g. by conformational changes due to the effected binding, the signals within signal cascades triggering a response of the cell and thus alter their features.

Itm2A was first found by Delersnijder et al. (1996) in a differential screen of a cDNA library from condyles from a mouse. The coded protein consists of 263 amino acids and represents an integral membrane protein of type II. It has a potential glycosylation site as well as a possible leucine zipper. The gene which consists of six exons is most strongly expressed in bone forming tissues and represents a marker for the differentiation cartilage/bone. Itm2A is member of a new gene family, consisting of three members. Between human and mouse the individual members of the family are high-conserved in each case. The conservation among the individual members only amounts to about 40% wherein predominantly the C-terminus is conserved, but not the N-terminus. The leucine zipper motive is only found for Itm2A, otherwise the proteins of the family do not contain known sequence motives.

### EXAMPLE 2

### Identification of S231

In the differential screen with the forward probe in comparison to the reverse probe the subtractive clone S231 showed a >5-fold stronger signal and was, thus, selected for sequencing. The sequence of a clone S231 is indicated as SEQ ID NO: 6. At BLAST homology searches the sequence S231 showed the highest homology to EMP1.

First, an expression pattern for S231 was prepared with the primers S231.1 (5′ CCA TAA CTC TTT CAC GCA ACT G 3′=-SEQ ID NO: 7) and S231.1R (5′ ACA ACA GAG GAG TTG GCT GTT T 3′=SEQ ID NO: 8) as described. GAPDH was used as control (see FIG. 3).

This semi-quantitative expression pattern shows that S231 is more strongly expressed in BMEC than it is in AOEC and, therefore, confirm the result of differential hybridization. Moreover, the expression in BMEC is also clearly stronger than in cortex (brain), being a hint towards the specificity for BMEC in the brain. Only in the heart a strong expression can be seen, however, only weak in lungs, colon or brain, although a strong expression is described in the literature (brain only for rat) for this tissue. This poses the question whether S231 actually represents EMP1 from pig, or if it is another member of this gene family.

In order to clear this, the cDNA library (λTriplEx2) from BMEC was screened with S231 as a probe (radioactively labelled, standard method). Several clones were isolated, the two biggest clones of which each were partially sequenced from 5′. Both sequences again showed the highest homologies to EMP1, wherein the overlaps in each case were located in the 3′non-coding region.

For the investigation if S231 was really about EMP1 from pig, a RT-PCR was performed with BMEC with the primers hsEMP1.s1 (5′ GGT ATT GCT GGC TGG TAT CTT T 3′=SEQ ID NO.: 9) and hsEMP1.as1 (5′ ATG TAG GAA TAG CCG TGG TGA T 3′=SEQ ID NO: 10), which were derived from the coding region of human EMP1. The product obtained (ssEMP1) was cloned and sequenced. From the sequence the primer ssEMP1.1 (5′ GGT CTT TGT GTT CCA GCT CTT C 3′=SEQ ID NO:11) was derived. A second primer ssEMP1.1R (5′ TTC TCA GGA CCA GAT AGA GAA CG 3′=SEQ ID NO: 12) was derived from a section of absolute congruence between the coding sequence of human EMP1 and EST F23116 from pig.

With these two primers ssEMP1.1/ssEMP1.1R an expression pattern was prepared as described above (cf. FIG. 4).

The expression patterns with the primers from clone S231 and from ssEMP1 are in fact similar, however, by no means identical. Therefore, it is to be postulated that S231 represents another member of the pmp-22/emp/mp20 gene family.

Both expression patterns were verified by Northern blot analyses wherein the clone S231 (FIG. 5A) and the PCR product hsEMP1.s1/hsEMP1.as1 (EMP1) (FIG. 5B), respectively, was used as a probe (cf. FIG. 5).

In the Northern blot the specificity for BMEC becomes even more clearly. This expression in BMEC and therefore at the BBB is hitherto not described.

Noticable is the in the Northern blot stronger expression in plexus (here, however, about the 2-3-fold amount of RNA was applied) and colon, whereas the expression by RT-PCR was stronger in the heart. Further is the ratio of the two transcripts in BMEC clearly different, depending on the probe used. With S231 the ratio of bigger transcript to smaller transcript is approximately the same, whereas with EMP1 as probe the smaller transcript appears significantly stronger. In comparison to the expression data of EMP1 in the literature it is noticeable that S231 from pig has different transcript sizes than EMP1 from human and mouse and that, furthermore, the expression pattern partly strongly departs from the literature data regarding EMP1 from different species. The discrepancies on the level of transcription show that the clone S231 as described herein does not represent EMP1, but is as S231 another member of this gene family. Possibly in humans only one gene EMP1 exists, which is regulated by two promoters, and in pig, this task is, however, taken over by two separate genes—EMP1 and S231.

In order to obtain the complete coding region of S231 from pig, the cDNA library from BMEC was screened in pTriplEx2 with EMP1 as ClonCapture probe. At this, several positive clones were isolated that contained the complete coding region. These were now sequenced and the protein sequence was deduced therefrom (SEQ ID NO: 13 and SEQ ID NO: 14).

The identity of S231 from pig to human EMP1 is only 78% on the level of amino acids, and is 76% to mouse. This further supports the thesis that S231 does not represent EMP, since normally proteins are 85-95% identical between man and pig, (cf. FIG. 7).

In order to obtain hints towards a possible role of S231 at the BBB the expression pattern in cultivated BMEC was investigated in comparison to known BBB markers and GAPDH, respectively, as described in Example 1 (cf. FIG. 8). These data show a quick decrease of the expression of S231 as was so described for known BBB-markers. A housekeeping gene such as GAPDH, however, does not show regulation.

The Western blot analysis for S231 which was performed according to the instruction given above confirmed the results which were obtained on the level of RNA. In BMEC, but not in AOEC, a strong expression of the protein can be noticed. Moreover, the Western blot shows that S231 predominantly occurs in the membrane fraction. In cultivated BMEC the expression decreases, but a protein having a lower molecular weight can increasingly be detected. Possibly, this is a matter of two different homo- and heterodimers, respectively of S231. The Western Blot is shown in FIG. 6.

The data clearly indicate a function of S231 at the BBB. Considering the described role of the protein during the differentiation of other cell types one can conclude that S231 is responsible for the special differentiation state of endothelial cells at the BBB and possibly represents a cell adhesion molecule or a channel (membrane domains most strongly conserved).

### EXAMPLE 3

### Identification of S012

In the differential screen with the forward probe in comparison to the reverse probe the subtractive clone S012 showed a >5-fold stronger signal and was thus selected for sequencing. The sequence of clone S012 is indicated in SEQ ID NO: 15. Based on the sequence S012 could unambiguously assign to the human hypothetical protein FLJ13448.

First, an expression pattern was prepared for S012 with the primers S012.s1 (5′ GTA TCG GGA GTG GAG GAT TAC A 3′=SEQ ID NO: 16) and S012.as1 (5′ CCC GAG GTA TAT TTG TTT CTG G 3′=SEQ ID NO: 17), as described above. GAPDH was used as a control (expression pattern not shown).

This semi-quantitative expression pattern shows that S012 is more strongly expressed in BMEC than it is in AOEC and, therefore, confirms the result of the differential hybridization. Moreover, the expression in BMEC is also clearly stronger than in cortex (brain), being a hint towards the specificity for BMEC in the brain. Only in the heart a strong expression can be seen. The full length cDNA of porcine S012/FLJ13448 was obtained by overlapping 5′ and 3′ RACE-PCR and is shown, together with a protein sequence in SEQ ID NO: 18 and SEQ ID NO:19.

The expression pattern was verified by Northern blot analysis. The full length clone FLJ13448/S012 (SEQ ID NO: 18) served as a probe for this (cf. FIG. 9).

In the Northern Blot the specificity for BMEC becomes even more clearly. The expression in BMEC and therefore at the BBB is hithereto not described. S012 is homologous to the human hypothetic protein FLJ13448 and the respective homologue from mouse (XM_{—}129724). A homology comparison of human, murine and porcine FLJ13448/S012 is depicted in FIG. 10. The peptides serving as signal peptides and being cleaved off are printed in italics in each case.

The low conservation of the N-terminal 60 amino acids and the high homology of the C-terminus, respectively, is striking. Probably, the N-terminus represents a signal peptide responsible for the correct localization of a protein within the cell. Bioinformatical investigations show a mitochondrial localisation of the protein in the cell. The function of the protein is to be attributed to the strongly conserved C-terminus.

In order to obtain hints to a possible role of a FLJ13448/S012 at the BBB, the expression pattern was investigated in cultivated BMEC in comparison to known BBB-markers and GAPDH, respectively, as described in Example 1 (cf. FIG. 11).

These data clearly point towards a role of FLJ13448/S012 of the BBB. The strong decrease of the expression in cultivated BMEC speaks for a correlation of FLJ13448/S012 with differentiation state of the cells.

### EXAMPLE 4

### Identification of NSE2

The sample material was prepared as described above under the section “Identification of BBB-specific proteins via 2D differential gel electrophoresis”.

The differential spot 1.1.0.1.10.37 resulted in the following peptide masses in the MALDI TOF analysis: 861.499; 878.47; 975.50; 1056.61; 1132.53; 1198.71; 1216.71; 1227.53; 1347.69; 1430.76; 1438.69; 1516.71; 1623.79; 1790.87; 1796.81; 1935.93; 1954.05; 2081.02; 2231.07; 2375.08; 2577.09; 2613.1.

Spot 1.1.0.1.10.37 was identified to be NSE2 by the database query with profound in the NCBI database. Human NSE2 has a calculated molecular weight of 34.5 kDa and a pi-value of 5.4 which both is in good agreement with the observed location of the spot 1.1.0.1.10.37 in the 2D gel. The peptide masses marked bold and underlined could be allocated as being identical to the human sequence. In FIG. 12 is depicted how peptide masses cover the human protein sequences.

First, an expression pattern for NSE2 was prepared with the primer ssNSE2.s1 (5′ CGC GTG GTG AAT GAT CTG TA 3′=SEQ ID NO: 20) and ssNSE2.as1 (5′ CTC CAT GAT CAG GTC CTC CAG 3′=SEQ ID NO: 21) as described. GAPDH was used as a control.

This semi-quantitative expression pattern shows that the expression of the NSE2 is the highest in the heart, followed by BMEC and Cortex (not shown). This result was confirmed by Northern blot analysis (cf. FIG. 13). For hybridization, the partial cDNA sequence of NSE2 from pig was used (SEQ ID NO: 22 and SEQ ID NO: 23) (partial CDS 1-192 encodes C-terminus), which was obtained by 3′RACE-PCR.

In order to obtain hints towards a possible role of NSE2 at the BBB, the expression pattern was investigated in cultivated BMEC in comparison to known BBB-markers and GAPDH, respectively, as described in Example 1. The result is shown in FIG. 14. These data show a quick decrease of the expression of NSE2, and, therefore, indicate a function of NSE2 at the BBB.

FIG. 15 shows a homology comparison of human NSE2 and NSE1.

Potential phosphorylation sites are depicted in pale font. Underlined is a possible tyrosine kinase domain (ProSite Pattern Match PS00109), wherein the active residue is depicted in bold. FIG. 16 shows the distribution of PEST-domains in NSE2. PEST sequences are Pro, Glu, Ser and Thr rich regions in proteins, responsible for short half-life of such proteins in the cell in that they control the ubiquitinylation of these proteins. Phosphorylation of certain Ser or Thr residues in the PEST regions (light grey) is important for the recognition of processing via the ubiquitin proteasome pathway.

Positions 81-163 in human NSE2 show homologies to the NLP/P60 family (pfam-domain 00877.4), which was found in several lipoproteins but was not attributed to a function.

Also this target was investigated under ischemic conditions. At this it became apparent that NSE2 is reduced in its expression in BMEC under ischemia (cf. FIG. 17). This speaks for an involvement of NSE2 in diseases connected with ischemic conditions such as stroke, cardiac infarction and tumor associated conditions such as at a glioblastoma. The expression pattern of NSE can, therefore, be used as diagnostic marker for such diseases. Moreover, a causative therapy can be based upon a modulation of the expression of NSE2.

### EXAMPLE 5

### Identification of DRG-1

The sample material was prepared as described above under the section “Identification of BBB-specific proteins via 2D differential gel electrophoresis”.

The differential spot 1.1.0.1.11.12 resulted in the following peptide masses in the MALDI TOF analysis: 789.45; 880.47; 890.50; 948.49; 1204.68; 1217.64; 1289.58; 1428.70; 1517.79; 1573.73; 1753.91; 2017.08.

Spot 1.1.0.1.11.12 was identified as hypothetical protein with the Accession Number CAB66619 by database queries with profound in the NCBI-database. The identical protein is also designated as dopamine responsive protein DRG-1, as LYST-interacting protein LIP5 and as HSPC228 in other entries of the database. The hypothetical protein CAB66619/DRG-1 has a calculated molecular weight of 33.8 kDa and a pI-value of 6.1 with both correlates very well with the observed location of the spot 1.1.0.1.11.12 in the 2D gel. The peptide masses marked in bold could be allocated as being identical to the human sequence. FIG. 18 shows how the peptide masses cover the human protein sequence.

A homology comparison between man (CAB66619) and mouse (XP-125508) shows very high homologies, in particular, in the region of aa 1-180. Bioinformatical approaches show a transmembrane domain and speak for that the N-terminus is localized intracellularly. The intracellular domain shows a conserved phosphorylation site, a glycosylation site is predicted extracellularly in the human sequence (cf. FIG. 19).

First, an expression pattern for DRG-1 was prepared with the CAB66619.s1 (5′ CGA GAC CCT GTG GTG GCT TAT TAC 3′=SEQ ID NO: 24) and CAB 66619.as1 (5′ CTG GTG TAT TAG CTG GAG CGT GTG 3′=SEQ ID NO: 25), as described. GAPDH was used as a control.

The semi-quantitative expression pattern (FIG. 20; which was confirmed by Northern blot analysis) shows that DRG-1 from pig is weaker expressed in BMEC than in AOEC and, therefore, is contradictory to the result of the 2D gels. Generally, DRG-1 is indeed expressed differently strong, but quite ubiquitous. Therefore, the difference found in the 2D gel must be attributed to a specific post-translational modification of DRG-1 in BMEC. Such a difference can e.g. occur due to the predicted phosphorylation site. Cell-specific phosphorylation can determine the activity of the proteins in that way.

In order to obtain hints towards a possible role of DRG-1 at the BBB, the expression pattern in cultivated BMEC was investigated in comparison to known BBB-markers and GAPDH, respectively, as described in Example 1 (cf. FIG. 21). These data show a clear decrease of the expression of DRG-1 and, therefore, indicate a function of DRG-1 at the BBB.

SEQ ID NO: 26+27 show the partial cDNA sequence of DRG-1 from pig (CDS1-585, internal section).

### EXAMPLE 6

### Identification of TKA-1

The sample material was prepared as described above under the section “Identification of BBB-specific proteins via 2D differential gel electrophoresis”.

The differential spot 1.1.0.1.6.30 resulted in the following peptide masses in the MALDI TOF analysis: 776.44; 847.47; 900.50; 916.46; 976.52; 1048.58; 1085.61; 1127.66; 1137.55; 1167.67; 1180.68; 1212.69; 1234.69; 1291.67; 1301.67; 1303.69; 1338.72; 1350.70; 1370.65; 1419.70; 1423.77; 1434.79; 1440.79; 1456.76; 1466.76; 1467.71; 1483.77; 1547.78; 1558.85; 1665.90; 1714.96; 1716.90; 1740.80; 1762.90; 1838.92; 1897.99; 2025.11; 2054.06; 2234.15; 2243.20; 2244.18.

Spot 1.1.0.1.6.30 was identified as TKA-l through the data-base queries with MSFIT in the NCBI-database. The peptide masses marked in bold and underlined could be allocated as being identical to the human sequence. In FIG. 22 is shown how the peptide masses cover the human protein sequence.

In the database, 3 isoforms of TKA-1 can be found which have the following calculated masses and pi-values: CAA90511 with 49.3 kDA/pI 6.7, BAA33216 with 37.4 kDA/pI 7.9, AAB53042 with 36.2 kDA/pI 8.2. The location in the 2D gel clearly speaks against the large isoform. Thus, the BAA33216 isoform was clearly found here experimentally, since in the protein Accession Number AAB53042 the peptide DGSAWKQDPFQ (in italics in FIG. 22) is missing, which, however, was partially (bold) detected within a trypsin fragment at the MALDI analysis.

The alignment of TKA-1 between man, mouse and rat shows a very high conservation. TKA-1 has two-PDZ domains which mediate protein-protein-interactions. In these PDZ domains several potential phosphorylation sites are located, whereby the interactions with other proteins are possibly regulated. Also a potential N-glycosylation site is conserved.

At first, an expression pattern for TKA-1 was prepared with the primers ssSLC9A3R2.s1 (5′ AAA AGG CCC CCA GGG TTA CG 3′=SEQ ID NO: 28) and ssSLC9A3R2.as1 (5′ GGA GTG GGC AGC AGG TGA GC 3′=SEQ ID NO: 29). GAPDH was used as a control.

The expression pattern was verified by Northern blot analysis. At this, the 550 bp PCR product ssTKA-1.ctg between the two primers ssTKA-1ctg.s1 (5′ TTA ACC TGC ACA GCG ACA AGT 3′=SEQ ID NO: 30) and ssTKA-1ctg.as1 (5′ TTG CTG AAG ATC TCA CGC TTC 3′=SEQ ID NO: 31) served as a probe.

The Northern blot (FIG. 23) shows that TKA-1 is expressed the strongest in BMEC and that three different transcripts occur in BMEC. The expression is comparably strong in lungs, here, however, the small transcript is missing completely. Up to now, no connection of TKA-1 to the BBB and neither to endothelial cells is described in the literature.

In order to obtain hints towards a possible role of TKA-1 at the BBB, the expression pattern was investigated in cultivated BMEC in comparison to known BBB-markers and GAPDH, respectively, as described in Example 1 (cf. FIG. 24). These data show a clear reduction of the expression of TKA-1 and, therefore, point to a function of TKA-1 at the BBB.

The target TKA-1 was investigated according to the instruction set forth above also as regards its expression under ischemia. At this, it showed that this target is strongly decreased in the expression in BMEC under ischemia. This speaks for a functional involvement of TKA-1 in diseases that come along with ischemic conditions such as stroke, cardiac infarction and tumor associated conditions such as at the gioblastoma. The investigation of the expression of TKA-1 can, therefore, be used as a diagnostic marker in such diseases. The target TKA-1 is a suitable starting point for causative therapies against the diseases mentioned above as well.

The expression pattern of TKA-1 in BMEC under ischemia compared with the control is shown in FIG. 25. BMEC were cultivated once under ischemia conditions (“ischemia”) and once under normal conditions (“control”) as described in the method section. Subsequently, the expression of target in both samples was measured relatively to 18S rRNA. The value obtained was set to 100% for the controls and the ischemia samples were referred thereto.

The Western blot analysis for TKA-1 confirmed the results obtained on the level of RNA. In BMEC a strong expression of the protein can be recognized, but barely in AOEC. Moreover, the Western blot showed that TKA occurs primarily membrane-associated and in the nucleus. In cultivated BMEC the expression very quickly decreases and is not detectable any more already in the first passage. The Western blot analysis of TKA-1 is shown in FIG. 26. SEQ ID NO: 32 and SEQ ID NO: 33 show the partial cDNA sequence of TKA-1 from pig (partial CDS 1-741 encodes the C-terminus).

### EXAMPLE 7

### Identification of S064/ARL8

In the differential screen with the forward probe in comparison to the reverse probe the subtractive clone S064 shows a >5-fold stronger signal and, therefore, was selected for sequencing. The sequence of the clone S064 is listed as SEQ ID NO: 35. On basis of this sequence S064 could not be allocated to a known gene. BLAST searches resulted in a significant homology to the DKFZ cDNA clone p43401317, which, however, obviously does not contain a coding region.

In order to identify the corresponding protein, a cDNA library from BMEC from pig was screened with the subtractive clone S064. At this, two independent clones were identified. The sequence of the longest clone S064.3 is listed as SEQ ID NO: 36. By BLAST searching this sequence could not be allocated to a known gene, either.

However, the sequence of clone S064.3 could be localized to the region 10p12 by homology comparisons in the human genome. The next gene in the same orientation at this locus is ADP-ribosylation-like factor 8 (ARL8). In order to check if S064 represents a new 3′ end of ARL8, a link PCR was performed. For this, the primers hsARL8.s1 (5′ TAA TGC AGG GAA AAC CAC CAT TCT 3′, SEQ ID NO: 37) and S064.3R (5′ AAC CAA GAG ACA TGT TGG CAC T 3′, SEQ ID NO: 38) were employed with RNA from BMEC in a OneStep RT-PCR. For checking the product specificity, the product from the OneStep RT-PCR was diluted 1:1,000 and employed in a nested PCR with the primers hsARL8.s2 (5′ ATA GCA TTG ACA GGG AAC GAC T 3′, SEQ ID NO: 39) and S064.GSP2 (5′ CTG CTA GAT TCA AGT CAT CAT GC 3′, SEQ ID NO: 40). The product obtained at this was cloned and sequenced. The sequence obtained clearly confirmed that the subtractive clone S064 represents the gene ARL8.

The complete coding cDNA sequence of ARL8 was obtained with the aid of a OneStep RT-PCR with RNA from BMEC and the primers S064cds.s1 (5′ C.TC GTG ATG GGG CTG ATC TTC 3′, SEQ ID NO: 41) and S064cds.as1 (5′ ATC TCA CAC CAA TCC GGG AGG T 3′, SEQ ID NO: 42). The coding sequence ARL8 from pig is indicated as SEQ ID NO: 43, the protein coded thereby is shown in SEQ ID NO: 44. The protein ARL8 is 100% identical to ARL8 from man and mouse. This high degree of conservation speaks for an important role of this protein. The cDNA sequence of ARL8 (pig) has 95% and 92%, respectively, homology in the coding region to the respective sequence from man and mouse, respectively.

An expression pattern was prepared for S064 with the primers S064.s1 (5′ AAG CCT GAA GCT TGA TGG ATA A 3′, SEQ ID NO: 45) and S064.as1 (5′ CAA TTA CAG CTT TGC TCC TGT G 3′, SEQ ID NO: 46), as described. 18S rRNA was used as a reference. Both primers S064cds.s1/as1 were derived by means of the human sequence due to the high homology between man and pig (e.g. of the product of the link PCR). Next to the general criteria of primer designing it was taken into account that the two primers flanked the complete coding sequence: That way, primer S064cds.s1 in position 7-9 contains the ATG start codon and position 22 in primer S064cds.as1 represents the first base of the stop codon. The expression pattern is shown in FIG. 27.

The expression pattern was repeated with another primer pair from the coding region of ARL8: ARL8cds.s1 (5′ ATA GCA TTG ACA GGG AAC GAC T 3′, SEQ ID NO: 47) and ARL8cds.as1 (5′ GAA CTG AGG GTG AGG TAT TTG G 3′, SEQ ID NO: 48). The expression pattern is shown in FIG. 28.

Moreover, the expression pattern was verified by Northern blot analysis. At this, clone S064 served as a probe. The result is shown in FIG. 29.

All three experiments show a very high specificity of ARL8 for BBB and, therefore, for the blood-brain barrier. This expression in BMEC and at the BBB, respectively, is hitherto not described. This high specificity indicated a very important role of ARL8 at the BBB.

In order to obtain hints towards the motor function of ARL8 at the BBB, the expression pattern was examined in cultivated BMEC in comparison to known BBB markers. The result is shown in FIG. 30.

The data show a quick decrease of the expression of ARL8 in cultivated BMEC and, therefore, clearly indicate an actual function of ARL8 at the BBB.

ARL8 belongs to the RAS super family of regulatory GTPases. These are involved in a multitude of processes such as cell growth signal transduction, organisation of the cytoskeleton and regulation of the membrane trafficking (exocytosis and endocytosis). ARL8 was first described by Sebald et al. 2003, who, however, could not show expression in the adult brain. The present example for the first time shows the actual expression of ARL8 at the BBB. This confirms the high BBB-specificity of this protein. The outcome of this is that ARL8 is responsible to the special differentiation state of endothelial cells at the BBB and, therefore, contributes to the efficiency of the BBB.

### EXAMPLE 8

### Identification of 5G9/PNOV1

In the differential screen with the forward probe in comparison with the reverse probe the subtractive clone 5G9 showed a >5-fold stronger signal and, therefore, was chosen for a sequencing. The sequence of clone 5G9 is listed as SEQ ID NO: 49. Based on this sequence, 5G9 could be allocated to a human transcript (No. BC039195, NCBI-database) which codes for a new protein HSNOV1 (AAH39195). In this database entry describing a mRNA molecule the open reading frame and the hypothetical protein resulting therefrom are indicated as annotation. This is not a matter of experimental data but of computer-based predictions. The deduced protein shows no similarity to known proteins and was, therefore, referred to as novel protein.

An expression pattern for 5G9 was prepared with the primers 5G9.1 (5′ TGT ATA TGT GGG ACA GCC ATC A 3′, SEQ ID NO: 50) and 5G9.1R (5′ GTC CGA GCA GGA TAT ACT TCC-A 3′, SEQ ID NO: 51), as described. 18S rRNA was used as a reference. The primer pair for determining the expression pattern was deduced according to general rules: melting temperature of the primers of 55-75° C.; approx. similar melting temperature of the two primers; 18-26 bases in length; optimal GC content 40-60%; avoiding of hairpins loops; avoiding of homo and heterodimer formation; product size 100-300 bp. The expression pattern is shown in FIG. 31.

The expression pattern shows that 5G9 is formed predominantly at the BBB, in the colon and in the kidney. In the brain, the expression seems to be specific for BMEC. This expression in BMEC and at the BBB, respectively, is hithereto not described.

In order to identify the corresponding protein from pig, starting from the sequence of the clone 5G9, the complete cDNA PNOV1 from pig was isolated by 5′ and 3′RACE-PCR (SEQ ID NO: 52). This transcript from position 480-1466 encodes a protein with SEQ ID NO: 53. The homology comparison between HSNOV1 and PNOV1 is shown in FIG. 32. The homology between HSNOV1 and PNOV1 is 94%. However, it is noticeable that PNOV1 is N-terminally shortened for 47 amino acids in comparison to HSNOV1. This sequence in HSNOV1 possibly presents a signal sequence, which is cleaved off later.

The protein HSNOV1 does not show any significant homologies to other known proteins. Bioinformatical analyses showed 8 potential transmembrane domains (cf. FIG. 33).

Also, several domains (e.g. InterPro-domain ipr002657) could be found, which indicate to a function as a transporter.

These data support that PNOV1/HSNOV1 resemble a new transporter at the BBB, which also occurs in the colon and in the kidney, two tissues having high transport activities for many substances.

### EXAMPLE 9

### Identification of 5E7/TSC-22

In the differential screen with the forward probe as compared to the reverse probe the subtractive clone 5E7 shows a >5-fold stronger thickness and was, therefore, chosen for sequencing. The sequence of clone 5E7 is listed as SEQ ID NO: 54. Based on this sequence, 5E7 could clearly be identified as transforming growth factor beta-stimulated protein TSC-22.

Clone 5E7 represents the 3′ end of the transcript TSC-22. In order to obtain the complete cDNA from pig, a 5′RACE-PCR was performed. The product of this PCR was cloned and sequenced. The complete cDNA sequence from TSC-22 from pig is listed as SEQ ID NO: 55. Here, the coding region is located from position 243-677. The protein corresponding hereto is listed as SEQ ID NO: 56. The porcine protein is 100% identical to the already human protein TSC-22, which speaks for a special importance of this protein.

An expression pattern was prepared for 5E7 by Northern-blot analysis as described. At this, the subtractive clone 5E7 served as a probe (cf. FIG. 34).

The experiment showed a strong expression of TSC-2 in BMEC in comparison to the total brain and, therefore, shows specificity for the blood-brain barrier. This expression in BMEC and at the BBB, respectively, is hithereto not described.

In order to obtain hints towards a possible role of TSC-22 at the BBB, the expression pattern was examined in cultivated BMEC in comparison to known BBB-markers and 18S rRNA, respectively. For the quantitative PCR the primers 5E7.1 (5′ AAG AGG TGT GGC TTG TCT TTT A 3′, SEQ ID NO: 57) and 5E7.1R (5′ TTT TTC AAA GTA TTC AAC CAG CTC 3′, SEQ ID NO: 58) were used. The result is shown in FIG. 35.

The data show a quick decrease of expression of TSC-22 in cultivated BMEC and, therefore, clearly indicate a role of TSC-22 at the BBB. The strong decrease of expression in cultivated BMEC speaks for that TSC-22 is connected with the differentiation state of the cells.

The expression of TSC-22 in BMEC under ischemia was investigated in the same manner. The result is shown in FIG. 36.

The target TSC-22 is strongly diminished in its expression in BMEC under ischemia. This substantiates a possible functional role of TSC-22 in diseases connected with ischemic conditions. To these belong stroke, cardiac infarction (TSC-22 is also strongly expressed in the heart, see FIG. 34) and the conditions in a tumor, such as the glioblastoma. The investigation of the expression of TSC-22 can therefore also be used as diagnostic marker for these diseases. Based on this knowledge, therapeutic concepts can be developed for diseases which go along with a dysfunction of the BBB.

TSC-22 belongs to the class of transcription factors with leucine zipper (Kester et al., 1999). It is involved in signal transduction of TGF-beta, among others (Kawamata et al., 1998) and, therefore, plays a role during cell growth and cell differentiation.

The outcome of this is that TSC-22 is co-responsible for the differentiation state of BMEC.

Literature:

Altschul, S. F., Gish, W., Miller, W., Myers, E. W., and Lipman, D. J. (1990): “Basic local alignment search tool”, J. Mol. Biol. 215, 403-410.
Birnboim, H. C. and Doly, J. (1979): “A rapid alkaline extraction procedure for screening recombinant plasmid DNA”, Nucl. Acids Res. 7: 1513-1522.
Cserzo et al. (1997): “Prediction of transmembrane alphahelices in procariotic membrane proteins: the dense alignment surface method”, Prot. Eng. 10: 673-676.
Deleersnijder, W., Hong, G., Cortvrindt, R., Poirier, C., Tylzanowski, P., Pittois, K., Van Marck, E., and Merregaert, J. (1996): “Isolation of markers for chondro-osteogenic differentiation using cDNA library subtraction. Molecular cloning and characterization of a gene belonging to a novel multigene family of integral membrane proteins”, J. Biol. Chem. 271, 19475-19482.
Kawamata, H., Nakashiro, K., Uchida, D., Hino, S., Omotehara, F., Yoshida, H., and Sato M. (1998): ”Induction of TSC-22 by treatment with a new anti-cancer drug, vesnarinone, in a human salivary gland cancer cell”, Brit. J. Cancer 77: 71-78.
Kester, H. A., Blanchelot, C., den Hertog, J., van der Saag, P. T., and van der Burg, B. (1999): “Transforming growth factor-□-stimulated clone-22 is a member of a family of leucine zipper proteins that can homo- and hetrodimerize and has transcriptional repressor activity”, J. Biol. Chem. 274: 27439-27447.
Li, J. Y., Boado, R. J., and Pardridge, W. M. (2001): “Blood-brain barrier genomics”, J. Cereb. Blood Flow Metabol. 21, 61-68.
Marvin, K. W., Fujimoto, W., Jetten, A. M. (1995): “Identification and characterization of a novel squamous cell-associated gene related to PMP22”, J. Biol. Chem. 270, 28910-28916.
Pearson, W. R. and Lipman, D. J. (1988): “Improved Tools for Biological Sequence Analysis”, PNAS 85, 2444-2448.
Sambrook, J., Fritsch, E. F., and Maniatis, T., in Molecular Clonin: A Laboratory Manual. Cold Spring Harbor Laboratory Press, NY, Vol. 1, 2, 3 (1989).
Sebald, E., Krueger, R., King, L. M., Cohn, D. H., and Krakow, D. (2003): “Isolation of a new member of the ADP-ribosylation like factor gene family, ARL8, from a cartilage cDNA library”, Gene 311: 147-151.
Shevchenko A., Sunyaev S., Loboda A., Shevchenko A., Bork P., Ens W. and Standing K. G. (2001); “Charting the Proteomes of Organisms with Unsequenced Genomes by MALDI-Quadrupole Time-of-Flight Mass Spectrometry and BLAST Homology Searching”; Anal. Chem. 73: 1917-1926.

1. A method for identifying the presence of a BBB-specific protein or fragment thereof in endothelial cells of brain capillaries, comprising
a) endothelial of brain capillaries freshly isolated from brain are conventionally pre-purified by enzymatic digestion,
b) the digest obtained in step a) is treated with a lysis buffer that essentially destroys erythrocytes and apoptotic cells present and maintains at least 70% of the endothelial cells of brain capillaries in vital form,
c) the product obtained in step b) is optionally purified further,
d) a subtractive cDNA library is prepared from the endothelial cells of brain capillaries and a subtractive tissue,
e) a cDNA subtraction is performed using one or more differential hybridization(s),
f) clones from the subtractive cDNA library are verified by differential hybridization with respect to their respective expression,
g) the cDNA sequence is completed for the BBB-specific clones from the subtractive cDNA library and
h) the expression pattern of the investigated clones is compared between fresh and cultured endothelial cells of brain capillaries and, that way, the presence of BBB-specific proteins or fragments thereof is identified.

2. The method according to claim 1, wherein the lysis buffer in step b) has the following composition:

| | | | | | |
|---|---|---|---|---|---|
| Na⁺ | 30.0 | mM | to | 60.0 | mM |
| K⁺ | 5.0 | mM | to | 7.5 | mM |
| NH₄⁺ | 80.0 | mM | to | 100.0 | mM |
| Ca²⁺ | 1.0 | mM | to | 2.0 | mM |
| Mg²⁺ | 6.0 | mM | to | 9.0 | mM |
| Cl⁻ | 125.0 | mM | to | 175.0 | mM |
| HCO₃⁻ | 4.5 | mM | to | 6.5 | mM |
| H₂PO₄⁻ | 0.5 | mM | to | 2.5 | mM |
| SO₄²⁻ | 0.3 | mM | to | 0.6 | mM |
| HPO₄²⁻ | 0.4 | mM | to | 0.7 | mM |
| Glucose | 1.5 | mM | to | 3.0 | mM |

3. The method according to claim 2, wherein the lysis buffer has the following composition:

| | | | | | |
|---|---|---|---|---|---|
| NaCl | 30 | mM | to | 50 | mM |
| KCl | 4.5 | mM | to | 5.5 | mM |
| NH₄Cl | 80 | mM | to | 100 | mM |
| CaCl₂ | 1.0 | mM | to | 2.0 | mM |
| MgCl₂ | 0.6 | mM | to | 0.8 | mM |
| MgSO₄ | 0.3 | mM | to | 0.6 | mM |
| NaHCO₃ | 4.5 | mM | to | 6.5 | mM |
| NaH₂PO₄ | 0.2 | mM | to | 0.45 | mM |
| Na₂HPO₄ | 0.4 | mM | to | 0.65 | mM |
| KH₂PO₄ | 0.1 | mM | to | 0.15 | mM |
| Glucose | 1.5 | mM | to | 3.0 | mM |

4. The method according to claim 1 wherein the subtractive tissue in step f) are aortic endothelial cells.

5. The method according to claim 1 wherein the complete cDNA sequence in step i) is prepared by screening cDNA libraries and RACE-PCR.

6. The method according to claim 1 the endothelial cells of brain capillaries are derived from man or pig.

7. A protein with BBB-specificity or a fragment thereof, obtainable according to a method according to claim 1.

8. The protein according to claim 7, wherein the protein comprises a sequence selected from SEQ ID NO: 5, SEQ ID NO: 14, SEQ ID NO: 19, or SEQ ID NO: 53.

9. A method for the identification of a presence of a BBB-specific protein or fragment thereof in endothelial cells of brain capillaries, comprising
a) endothelial cells of brain capillaries freshly isolated from brain are conventionally pre-purified by enzymatic digestion,
b) the digest obtained in step a) is treated with a lysis buffer that essentially destroys erythrocytes and apoptotic cells present and maintains at least 70% of the endothelial cells of brain capillaries in vital form,
c) the product obtained in step b) is optionally purified further,
d) the product obtained in step c) is solubilized in a suitable buffer,
e) an isoelectric focusing is performed,
f) the samples from the isoelectric focusing are separated in the second dimension according to the molecular weight,
g) differential spots are identified and isolated,
h) mass spectrometric analysis is performed with the isolate of g), and
i) an evaluation thereof is constructed via specific database analysis.

10. The method according to claim 9, wherein a lysis buffer in step b) has the following composition:

| | | | | | |
|---|---|---|---|---|---|
| Na⁺ | 30.0 | mM | to | 60.0 | mM |
| K⁺ | 5.0 | mM | to | 7.5 | mM |
| NH₄⁺ | 80.0 | mM | to | 100.0 | mM |
| Ca²⁺ | 1.0 | mM | to | 2.0 | mM |
| Mg²⁺ | 6.0 | mM | to | 9.0 | mM |
| Cl⁻ | 125.0 | mM | to | 175.0 | mM |
| HCO₃⁻ | 4.5 | mM | to | 6.5 | mM |
| H₂PO₄⁻ | 0.5 | mM | to | 2.5 | mM |
| SO₄²⁻ | 0.3 | mM | to | 0.6 | mM |
| HPO₄²⁻ | 0.4 | mM | to | 0.7 | mM |
| Glucose | 1.5 | mM | to | 3.0 | mM |

11. The method according to claim 10, wherein the lysis buffer has the following composition:

| | | | | | |
|---|---|---|---|---|---|
| NaCl | 30 | mM | to | 50 | mM |
| KCl | 4.5 | mM | to | 5.5 | mM |
| NH₄Cl | 80 | mM | to | 100 | mM |
| CaCl₂ | 1.0 | mM | to | 2.0 | mM |
| MgCl₂ | 0.6 | mM | to | 0.8 | mM |
| MgSO₄ | 0.3 | mM | to | 0.6 | mM |
| NaHCO₃ | 4.5 | mM | to | 6.5 | mM |
| NaH₂PO₄ | 0.2 | mM | to | 0.45 | mM |
| Na₂HPO₄ | 0.4 | mM | to | 0.65 | mM |
| KH₂PO₄ | 0.1 | mM | to | 0.15 | mM |
| Glucose | 1.5 | mM | to | 3.0 | mM |

12. A protein with BBB-specificity or a fragment thereof, obtainable according to a method according to claim 9.

13. The protein according to claim 12, wherein the protein comprises a sequence selected from SEQ ID NO: 23, SEQ ID NO: 27, SEQ ID NO: 33.

14-15. (canceled)

16. An agent for the diagnosis of diseases that are based on the dysfunction of the blood-brain barrier, characterized in that it comprises a protein according to claim 7.

17. The agent for the therapy of diseases which are based on a dysfunction of the blood-brain barrier, characterized in that it comprises a protein according claim 7.

18-20. (canceled)

21. A method for identifying the presence of a BBB-specific protein or fragment thereof in endothelial cells of brain capillaries, comprising:
a) purifying endothelial of brain capillaries,
b) treating the digest obtained in step a) with a buffer that can essentially destroy erythrocytes and apoptotic cells present and maintain at least about 70% of the endothelial cells of brain capillaries in vital form,
c) optionally purifying further the product obtained in step b) is optionally purified further,
d) preparing a subtractive cDNA library from the endothelial cells of brain capillaries and a subtractive tissue,
e) performing a cDNA subtraction,
f) verifying clones from the subtractive cDNA library,
g) completing the cDNA sequence for the BBB-specific clones from the subtractive cDNA library and
h) comparing the expression pattern of the investigated clones between fresh and cultured endothelial cells of brain capillaries and thereby identifying the presence of BBB-specific proteins or fragments thereof.

22. A method for diagnosis of a disease or condition associated with an ischemic condition, comprising use of one or more sequences that comprise SEQ ID NO: 4, SEQ ID NO: 6, SEQ ID NO: 15, SEQ ID NO: 22, SEQ ID NO: 23, SEQ ID NO: 26, SEQ ID NO: 32, SEQ ID NO: 35, SEQ ID NO: 36, SEQ ID NO: 43, SEQ ID NO: 49, SEQ ID NO: 52, SEQ ID NO: 54, SEQ ID NO: 55, for the preparation of an agent for the diagnosis of diseases which are connected with ischemic conditions.

23. The method according to claim 22 wherein the one or more sequences are used to diagnosis of stroke, myocardial infarction or tumor-associated conditions.

24. The method of claim 22 wherein the diagnosis is carried out via the control of the expression of one or more polypeptides encoded by the one or more sequences.

25. A method for transporting a substance through the blood-brain barrier comprising using a polypeptide of claim 7.

26. A method for diagnosis or therapy associated with blood-brain barrier dysfunction comprising use of a protein of claim 7.


SEQUENCE LISTING (70 sequences)

SEQ ID NO: 1 - DNA, 323 bp, Artificial Sequence
  Description of Artificial Sequence Synthetic clone S129 from BMEC from swine brain
ctgcagccgaggacaacactgattcgagccgtgacctaccggccgcgggaattcgatttatggtgaaaatcgccttcaatacacccgcagcggtgcaaaaagaggaggcgcagcaagacgtggaggccctcgtaagccatacggtccgtgctcagatcctgactggcaaggaactccaagttgccactaaggaaaaagagggcttctctgggagatgcatgcttactctcgtaggcctttccttcatcttggcaggacttattgttggtggagcctgcatttacaagtacttcatgcccaagagtaccatactaccatggaga

SEQ ID NO: 2 - DNA, 22 bp, Artificial Sequence
  Description of Artificial Sequence Synthetic primer
acctccattgttatgcctccta

SEQ ID NO: 3 - DNA, 22 bp, Artificial Sequence
  Description of Artificial Sequence Synthetic primer
gttgcctctcactcttgacaga

SEQ ID NO: 4 - DNA, 1598 bp, Sus sp.
  CDS (119)..(910)
  modified_base (1168): a, c, g, t, unknown, or other
gcggccgctagcataaagaaggtgattctaagcctagcgctatcttctcctagtccagcctgcagccgaggacaacactgattcgagccgtgacctaccggccgcgggaattcgatttatggtgaaaatcgccttcaatacacccgcagcggtgcaaaaagaggaggcgcagcaagacgtggaggccctcgtaagccatacggtccgtgctcagatcctgactggcaaggaactccaagttgccactaaggaaaaagagggcttctctgggagatgcatgcttactctcgtaggcctttccttcatcttggcaggacttattgttggtggagcctgcatttacaagtacttcatgcccaagagtaccatctaccatggagagatgtgcttctttgattctgcggaccctgcaaatttcctccaaggaggagagccctacttcctgcctgtgatggaagaggctgatattcgtgaagatgacaacattgcaatcattgatgtgcctgtccccagtttctctgatagtgaccctgcagcaattattcatgactttgaaaagggcatgactgcttacctggacttgctgctggggaactgctatctgatgcccctcaatacctccattgttatgcctcctaagtatctcgtggagctctttggcaaactggcacgtggcaaatacctccctcacgcttatgtggttcatgaagacctggttgctgtggaagagattcatgatgttagtaaccttggcatctttatttaccaactttgcaacaaccgcaagtctttccgccttcgtagaagagacctcttgctgggtttcaacaaacgtgccattgataagtgctggaagattagacacttccccaatgaatttattgttgagaccaagatctgtcaagagtgagaggcaacagaaaaagagtgtacttagtaataggaagtcaaagatttacaatatgacttcaatattaaagtgtgtaggacattcaagatatttactcatgcatttcctctattgcttatacttaaaaaaaagaaagaaaataaaaactactaaccattgcaaaaaaaaaaaaaaaaagtactagtcgacgcgtggccagaaactgaaatgaaatgatttttatgtttttccttttgaatttatagggtttatgttttnttgaatgcaatgtgaaggtgttggctaacatcctgacaatgaattccatcccttgtgtatatgtgtgtctttaaaagtaaaatyttcartcatatggtaaaacatgttttaaatttaaaatatttaaaattgttttcaacctttttgtgtagcgcttgtcaaatatcttaacattgtcttgttttgttttcattgtgtgcaactttcctgaatttagaaattaaatttttgcatttatgttaggtgttctgtaatagatatgacttatatgtgaaaaactttcataaagaagtcattttcactaatrcagtgactctcactggtaactgtattgtgaaatgcacaaaactgttttagtgctgaatgctataaggaatttaggttgtatgaattctacaatcctataataaattttaccatattcaaaaaa

SEQ ID NO: 5 - protein, 263 aa, Sus sp.
MVKIAFNTPAAVQKEEAQQDVEALVSHTVRAQILTGKELQVATKEKEGFSGRCMLTLVGLSFILAGLIVGGACIYKYFMPKSTIYHGEMCFFDSADPANFLQGGEPYFLPVMEEADIREDDNIAIIDVPVPSFSDSDPAAIIHDFEKGMTAYLDLLLGNCYLMPLNTSIVMPPKYLVELFGKLARGKYLPHAYVVHEDLVAVEEIHDVSNLGIFIYQLCNNRKSFRLRRRDLLLGFNKRAIDKCWKIRHFPNEFIVETKICQE

SEQ ID NO: 6 - DNA, 814 bp, Artificial Sequence
  Description of Artificial Sequence Synthetic clone S231 from BMEC from swine brain
acatttctttaggttcattctggttaaggggatgttcgagggtgggccaccaaattgtctgggctggggataaagcagttggcaagcaaaaactatgggatgatgaacttttcaatwatgatttaatgatcacatgagtatagaaagctgttttgagtgctgaaacagacttacctatcagatatatccaaaagagattctatgttaaaaagtcagactatgactggagtgaaccatgtattcccttgtcttttactttgtttctgtgacatttatgtttcatgtaacttgcattatggttgggtgggttgtcctagtactgtattttggcttcttctttaataggattgatatttcatatabtataattgtgaatattttgakacraatgtttataactctaggcatataaaaacagattctgattcccttcactgtgtgaatgttttctgttgaaaaaatggaggataaatatggatactaatgacactcattcctaattaagttttcaatcagtttgatttggataacttgcatttatccgagatattgagctactttctgataatgcatcaagcatttctaccataactctttcacgcaactgaatgttgttaagtatagttttatcttgctttaattaaacttcttaagcaaaaaaaaagaaacttcataagctaatacattagagaaaggttatgatcttgaatcnagaatggcttatggcattaaggaatgagatacttgtaaattttctttgaaacagccaactcctctgttgtgtcttcacaattcaaaagatatgcctcactgt

SEQ ID NO: 7 - DNA, 22 bp, Artificial Sequence
  Description of Artificial Sequence Synthetic primer
ccataactctttcacgcaactg

SEQ ID NO: 8 - DNA, 22 bp, Artificial Sequence
  Description of Artificial Sequence Synthetic primer
acaacagaggagttggctgttt

SEQ ID NO: 9 - DNA, 22 bp, Artificial Sequence
  Description of Artificial Sequence Synthetic primer
ggtattgctggctggtatcttt

SEQ ID NO: 10 - DNA, 22 bp, Artificial Sequence
  Description of Artificial Sequence Synthetic primer
atgtaggaatagccgtggtgat

SEQ ID NO: 11 - DNA, 22 bp, Artificial Sequence
  Description of Artificial Sequence Synthetic primer
ggtctttgtgttccagctcttc

SEQ ID NO: 12 - DNA, 23 bp, Artificial Sequence
  Description of Artificial Sequence Synthetic primer
ttctcaggaccagatagagaacg

SEQ ID NO: 13 - DNA, 483 bp, Artificial Sequence
  Description of Artificial Sequence Synthetic clone S231 from BMEC from swine brain
atgttggtgttactggctggtatctttgtggtccacatcgccactgtcgtcatgctgttcgtttgcaccattgccaatgtctgggtggtctcagatgcgggacaaggatctgtcggtctttggaaaaactgtaccagtgctggctgtactgataccctgttatacggcggtgaagatgccctcaagtcggtgcaggccttcatgatcctgtctatcatcttctctgtcgtctccctcgtggtctttgtgttccagctcttcaccatggagaaaggcaaccgcttcttcctctcgggagccaccatgctggtgtgctggctgtgcatcatggtgggggcctccgtctatactcatcattatgccaacagttctaaaaaccaatactcggcgagtcaccatggctattccttcatcctcgcctggatctgcttctgcttcagcttcatcatcggcgttctctatctggtcctgagaaagaaataa

SEQ ID NO: 14 - protein, 160 aa, Artificial Sequence
  Description of Artificial Sequence Synthetic clone S231 from BMEC from swine brain
MLVLLAGIFVVHIATVVMLFVCTIANVWVVSDAGQGSVGLWKNCTSAGCTDTLLYGGEDALKSVQAFMILSIIFSVVSLVVFVFQLFTMEKGNRFFLSGATMLVCWLCIMVGASVYTHHYANSSKNQYSASHHGYSFILAWICFCFSFIIGVLYLVLRKK

SEQ ID NO: 15 - DNA, 513 bp, Artificial Sequence
  Description of Artificial Sequence Synthetic clone S012 from BMEC from swine brain
acatagaattcaatcaagtgtaattcagaataatgtgtatattagcatatttacagtaatgggatgtcatcgctattgttagaatactgacatcacttttctgagcagaaattgaaactgtaaatttaaccttttaattatcacctcacctgaaaaggttggttgagatactcacgcagcatgtattatattaaccatgtcatgtttaagttattaaattcagattatttataacttattatcttagggcctgcctcatgtcttctagggtatttgagtaatcatcctatatttaaagttaaaactttgacttaaaaaacactgttaatgaaagttccctagcgcttttcttattttcaaattggtcttatgggtagtagtagagaattccatgctgttctgaggctagcttccaggtaaacagtgattttttttttctttttttctttctttcttggtgagtggtccagagttttaagctacttttctcaaagtttcaaccctttcccaggt

SEQ ID NO: 16 - DNA, 22 bp, Artificial Sequence
  Description of Artificial Sequence Synthetic primer
gtatcgggagtggaggattaca

SEQ ID NO: 17 - DNA, 22 bp, Artificial Sequence
  Description of Artificial Sequence Synthetic primer
cccgaggtatatttgtttctgg

SEQ ID NO: 18 - DNA, 1674 bp, Sus sp.
  CDS (40)..(774)
ccgtctgcctggtcccaraggcgcaccggcttcggtaacatgtttgtggcagctcggacaggccagagaaccttgagaaaggtggtctcgggatgccgtccaaaatcggcgacagcgactggagtcccggctcctgcgcaggggcctccgcggaacatcagatacttagcctcctgtggtatactgatgaacagaactcttccactgcattcctcatttttgcctaaggagatgtatgcaagaaccttcttcagaattgctgcaccattaataaacaaaagaaaagaatattcagagaggaggattataggatattctatgcaggaaatgtatgacgtagtatcgggaatggaagattacaagcattttgtgccttggtgcaaaaaatcagatgtaatatcaaggagatctggatactgcaaaacacgattagaaattgggtttccacccgtattggagcgctatacgtcagtagtaaccttggtgaaaccacatttggtaaaggcatcctgtgcagatgggaagctctttaatcacttagagactgtttggcgttttagcccaggtcttcctggctacccaagaacttgtactttggatttttcaatttcttttgaatttcgatcacttctgcactctcagcttgccacattgttttttgatgaagttgtgaagcagatggtagctgcttttgaaagaagagcatgtaaactgtatggtccagaaacaagtatacctcgggaattaatgcttcatgaagttcatcacacataagagaaaaggaaatggttgcctacttgtaactagtttattcacttttaggaagtgctttcatcattttgctytcagaaggcagaaagcatttgtcaaacacagctttgatataaacctgtactttgcacttggaatatggaaccacatgtacatagaattcaatcaagtgtaattcagaataatgtgtatattagcatatttacagtaatgggatgtcatcgctattgttagaatactgacatcacttttctgagcagaaattgaaactgtaaatttaaccttttaattatcacctcacctgaaaaggttggttgagatactcacgcagcatgtattatattaaccatgtcatgtttaagttattaaattcagattatttataacttattatcttagggcctgcctcatgtcttctagggtatttgagtaatcatcctatatttaaagttaaaactttgacttaaaaaacactgttaatgaaagttccctagcgcttttcttattttcaaattggtcttatgggtagtagtagagaattccatgctgttctgaggctagcttccaggtaaacagtgattttttttttctttttttctttctttcttggtgagtggtccagagttttaagctacttttctcaaagtttcaaccctttcccaggtactttgactactatttcagtaatgttgattgtgtgtcaagttttgtctacagcagtgggcaatagatgaaggaagtcggttgatatgtctccaacaccatgcattctgattttctatttattgtgtatactcactttcaataatgtatttccaactgatatttttgtaaacaaatcagtgtaaggactgaagtggtaacttaataaagttaatttgtttaaaaaataaaaaaaaaaaa

SEQ ID NO: 19 - protein, 244 aa, Sus sp.
MFVAARTGQRTLRKVVSGCRPKSATATGVPAPAQGPPRNIRYLASCGILMNRTLPLHSSFLPKEMYARTFFRIAAPLINKRKEYSERRIIGYSMQEMYDVVSGMEDYKHFVPWCKKSDVISRRSGYCKTRLEIGFPPVLERYTSVVTLVKPHLVKASCADGKLFNHLETVWRFSPGLPGYPRTCTLDFSISFEFRSLLHSQLATLFFDEVVKQMVAAFERRACKLYGPETSIPRELMLHEVHHT

SEQ ID NO: 20 - DNA, 20 bp, Artificial Sequence
  Description of Artificial Sequence Synthetic primer
cgcgtggtgaatgatctgta

SEQ ID NO: 21 - DNA, 21 bp, Artificial Sequence
  Description of Artificial Sequence Synthetic primer
ctccatgatcaggtcctccag

SEQ ID NO: 22 - DNA, 607 bp, Artificial Sequence
  Description of Artificial Sequence Partial cDNA sequence of NSE2 from swine
gaggacctgatcatggagaaacggcgcaacgaccagatagggcgcgccgcggtgctacaggagctggccacgcacctgcaccccgcggagccggacgagggcgacagcgacgccgcgcggactacgccgcctcccgggcgctcccaggcgccgggccaagaggaggaggaccgagaggcggtggtgcactgacaggcgagctgagtgcggagctgcgtgagggagcctttgcagcagccgctgccccctcccttctctccctccctcctccaccatcttctgggtcccaactgggctcctgggccatttggaaaacggagagttggcgaaaagcgctgccagctgtggcttgagtttgttatcttggacggaggaggaagagggagcagcttccatggacccctgatcactacttgaggagaattttcctgtggattcaactgactagctattgtgatgtaagcagtttgaggtgactggcccagcaggagtgagaagaatttatcttcagcataaacttcattattctacagtgtttcttcatttgcctgagaggtaaggatgctatgtagacagaaacaaaggaagaaaaaaaaaaaaaaaaaaa

SEQ ID NO: 23 - protein, 63 aa, Artificial Sequence
  Description of Artificial Sequence Partial amino acid sequence of NSE2 from swine
EDLIMEKRRNDQIGRAAVLQELATHLHPAEPDEGDSDAARTTPPPGRSQAPGQEEEDREAVVH

SEQ ID NO: 24 - DNA, 24 bp, Artificial Sequence
  Description of Artificial Sequence Synthetic primer
cgagaccctgtggtggcttattac

SEQ ID NO: 25 - DNA, 24 bp, Artificial Sequence
  Description of Artificial Sequence Synthetic primer
ctggtgtattagctggagcgtgtg

SEQ ID NO: 26 - DNA, 586 bp, Sus sp.
  CDS (1)..(585)
cgagaccctgtggtggcttattactgtcgtttatatgcaatgcaaactggaatgaagattgatagtaaaactcctgaatgtcgtaaatttttatcaaagctgatggatcagttagaagctcttaagaaacagttgggtgacaatgaagctgttactcaagaaatagttggttctgcccacttggagaattatgctttgaaaatgtttttatatgcagataatgaagatcgggctgggcgatttcataaaaacatgatcaagtccttctatactgcaagtcttttaatagatgtcataacagtgtttggagaactcactgatgaaaatgtgaaacacagaaagtatgcaaggtggaaggcaacatatattcataattgtttaaagaatggagggactcctcaagcaggtcctgtgggcattgaagaagataatgacatagaagaaaatgaagatgctggagcaacctctctgcccactcagccacctcagccatcatcttccacttatgacccaggcaacatgccatcgagcagctatactggaatacagattcctcccggtgcacacgctccagctaatacaccag

SEQ ID NO: 27 - protein, 195 aa, Sus sp.
RDPVVAYYCRLYAMQTGMKIDSKTPECRKFLSKLMDQLEALKKQLGDNEAVTQEIVGSAHLENYALKMFLYADNEDRAGRFHKNMIKSFYTASLLIDVITVFGELTDENVKHRKYARWKATYIHNCLKNGGTPQAGPVGIEEDNDIEENEDAGATSLPTQPPQPSSSTYDPGNMPSSSYTGIQIPPGAHAPANTP

SEQ ID NO: 28 - DNA, 20 bp, Artificial Sequence
  Description of Artificial Sequence Synthetic primer
aaaaggcccccagggttacg

SEQ ID NO: 29 - DNA, 20 bp, Artificial Sequence
  Description of Artificial Sequence Synthetic primer
ggagtgggcagcaggtgagc

SEQ ID NO: 30 - DNA, 21 bp, Artificial Sequence
  Description of Artificial Sequence Synthetic primer
ttaacctgcacagcgacaagt

SEQ ID NO: 31 - DNA, 21 bp, Artificial Sequence
  Description of Artificial Sequence Synthetic primer
ttgctgaagatctcacgcttc

SEQ ID NO: 32 - DNA, 1194 bp, Sus sp.
  CDS (1)..(741)
acggacgaggagctccgcaggcgccagctgacttgcaccgaggagatggcccagcgagggctgccgcctgcccttgacccctgggagccgaaggcggactgggcgcccgcaggcagcctcagcggtgaggccggccagaaggatgtcaacgggcccctgagggagctgcgcccaaggctctgccacctgcgaaaaggcccccagggttacgggtttaacctgcacagcgacaagtcccggcctggacagtacatccgctccgtggacccaggctcacctgctgcccactccggcctccgagcccaggaccgactcatagaggtgaacgggcagaatgtggaggggctgcggcacgcggaggtggttgcctgcatcaaggcgcgggaggacgaggcccggctgctggtggtggaccccgagacggatgtgtacttcaagcggctgcgggtcacacccacccaggagcacatggaaggtccactgtcatcacctgtcaccaatgggaccagctcagcccagctcaatggtggctccgtgtgctcgtcccgaagtgacctgcccggcttagacaaggacactgaggacagcagcacctggaagcgtgaccctttccaggagagtggcctccacctgagccccacggcggctggggccaaggagaaggcgagggccaccagggtcaacaagcgggcgccacagatggactggaaccggaagcgtgagatcttcagcaacttctgagaccccccaccctccgccgcagccgccgcctggtccccagccgggcctcctctgggcatggaccttgggccttgcccagagcgccccgagcctcagtggactgcagcgggggcaccttcgctcgctaagccgtggtggtcccaccaccccccatgaaccagcccgtgccccagtgagcccccgtcctgcccccttcccacggggtgctggggagcgggcagaggaagcccctgagacgggagggacagagacacccagagaggtgggctggggaggggaggttggggtgacccgccaggccgggcccttgctgctctgcctgggcctgctgacttaaaggaatttgtgttttggctttttttccaacacgagctctggctccacacatgtttccacttaataccagagcccccacccccatcccctcaggacgtgctctctaaataattgcaa

SEQ ID NO: 33 - protein, 246 aa, Sus sp.
TDEELRRRQLTCTEEMAQRGLPPALDPWEPKADWAPAGSLSGEAGQKDVNGPLRELRPRLCHLRKGPQGYGFNLHSDKSRPGQYIRSVDPGSPAAHSGLRAQDRLIEVNGQNVEGLRHAEVVACIKAREDEARLLVVDPETDVYFKRLRVTPTQEHMEGPLSSPVTNGTSSAQLNGGSVCSSRSDLPGLDKDTEDSSTWKRDPFQESGLHLSPTAAGAKEKARATRVNKRAPQMDWNRKREIFSNF

SEQ ID NO: 34 - protein, 63 aa, Sus sp.; identical to SEQ ID NO: 23

SEQ ID NO: 35 - DNA, 367 bp, Artificial Sequence
  Description of Artificial Sequence Synthetic clone S064 from BMEC from swine brain
acaataccaggggtcccccagagagatcctgttcataattttgtcctttttaacaccatttcatttgatcaagctgattagctaagatcttgttacagcatttgcagaaagcctgaagcttgatggataacaacagttttaaaccttaagaaatgacaagtataaatacagacacttcaatgtagttttacattctgaggcaagaaatatattatacagggcctgctgtttcctctttaatgctctaaaagcaccaatttatgttaaagatggcaatgtgtaattataatcattataatctgattagaccaaacacaggagcaaagctgtaattgcttttagtttttgtttttttaacatgctctgt

SEQ ID NO: 36 - DNA, 3071 bp, Artificial Sequence
  Description of Artificial Sequence Synthetic clone S064.3 from BMEC from swine brain
sctwtggcggggwatctcwaggacaaatwwwaatggaatwatctctggctggcactcatttaattcttaactatgtaaaacaacatgagtagaaaaaaatttagtggtattatgcctagaatagataggtgaattccattgatgtttatctttgaagaccagctttatgcgtgaacttttcatctgwggctttggatccaaaacatttcatgtccagttcagttctaaaggttcttttatattttgtcagggtagtctctttgagatacagcatgatgacttgaatctagcagaatattgtgctggctacctaaagaagtgggttcaaatcttaatttggccattaccttttgaccttagacagttactactgtttatggtcttccttctgtttttcccatgcagaggaacttaaacaaattatagagtgccaacatgtctcttggttttaaaatcgtgaatctattaaaatcccgaatctactaaaacactattaaaaactggaaaaaaaattcaactagggaaagacatgtaatatgaaatttatttttacctatcatttgattcccactttattatcytttcatttagtatatgaatacaatccaataagaaaatgaaggtcaactactgccactccacttaaattgaactaatagttaatgaagtgcaaaagagaaaataagccatattgctaagaagatgatatattaagctgctgataaaataccagtgtgtgttgaaaatactcttttagaagataccttgcttattttcctggcttttattaattggatggaaatggttagtttgatcagagtttattggctctagaggctgccccaaattgtagctctgtttgactttccagtattgaaagaatactggaaatgtcaatattttacaaatgtctgtacaaatctgaaagtagtttatatccatggttagttttttcagtaacgttccatccttattcatttagcattactgtaaagccaggttcccaagaagtattttctaagagttccaagtaaccacagctacatagagaaagccaataaaaacaaaactttttagctacttctctgtaaatttaaagtagaaaaaaaccagacctaaagtcagctttraatgtatgtggtctagtgaaatgtttgggaaatgtttatttggaggtttagaggcataccgaagcaggagtcaaaacaaagttggtggtaaagattaacatgaagtaaaaaaatcttcagtagaaaatagaaagtttgaatgaaaacaatgagttgtccccattcaaggcacttaaaatwactagaaaattctgtcttttactgtaattggatggcctatattatttctaatgtggccaaaggactaaagaccaatcaggtttctagaattggggagcgtagtcacatagaggcatcttttgcattttttaannnaccagtaatcttccttttccccttagaaakggagaaataaaatgttctgtacatatcttttggaatagaaagcaaaattctagaagaatggaagtatcctcttacaccaacttgtagttttaattgaaaaattacctcatttttcagtccatacggtgctttgctcgagtttgtggaatggtccaccatcccattaaaacccgcttcacccaagctgtatttcaaatatgcaaaattcacagctaagggatagcagtccttggaggttttgttttccttcactcgcgcttaccaccagcagagctaataacgtgatgtaccaggttgacatactgcttcattaaagcacatgggcaagtgtttagtcaatatttaattagtttaattaaaatcaaataagggaaaggaaaaacccttaagtttgattgagttacattatactgtgaatatatttccatctgtgttgataagacatcaaatgactatcagttgatattgattatacataatttatttgcatattctggccctattcatgagaggctataatcattttaatcttacattttccttcaggaaattcagggactctacagcccctattttgttctcttggagtaaawtgttcagtgtagtttatgaaaacttttcattttggttttaaaaaaggcttagctgctagttcattaaaagtgtgaaataaaatgatggttatgatttttccaattaatgttataaattttascstrtycrtcyrwkgtacagagcatgttaaaaaaacaaaaactaaaagcaattacagctttgctcctgtgtttggtctaatcagattataatgattataattacacattgccatcttaacataaattggtgcttttagagcattaaagaggaaacagcaggccctgtataatatatttcttgcctcagaatgtaaaactacattgaagtgtctgtatttatacttgtcatttcttaaggtttaaaactgttgttatccatcaagcttcaggctttctgcaaatgctgtaacaagatcttagctaatcagcttgrtcaaatgaaatggtgttaaaaaggacaaaattatgaacaggatctctctgggggacccctggtattgtacmkrmssgggsggaacygtctykmatgccacaaactgtgcgtcataatcccacccaaacaactgacatgtgtgtwattggttcaatacataagcattaataaaattaaaggaacaaattacttaaagcagtcacatcatcacttcctcaaagtggttyaaagcatgttcttctaaatggtggagttgtttaaagacatgttttaaattttgatagctttactactgtcataaaatgcttctatatgttaagtttaggttgctggtactcatgattttttacttctgcaattatgctgtaatgagttgcttgcatgcctacttacccaagtgaaaggatgctgtttgctctggaatgttcatcttttagacaggtttksgctcatttgcaatcatggtgcaatacagtgtaacattcatttgttttcagtcaatagttttatttttgtcmcaataaataattacttttccaaaaaaaaaaaaaaaaaaaaaaaaaaaaa

SEQ ID NO: 37 - DNA, 24 bp, Artificial Sequence
  Description of Artificial Sequence Synthetic primer
taatgcagggaaaaccaccattct

SEQ ID NO: 38 - DNA, 22 bp, Artificial Sequence
  Description of Artificial Sequence Synthetic primer
aaccaagagacatgttggcact

SEQ ID NO: 39 - DNA, 22 bp, Artificial Sequence
  Description of Artificial Sequence Synthetic primer
atagcattgacagggaacgact

SEQ ID NO: 40 - DNA, 23 bp, Artificial Sequence
  Description of Artificial Sequence Synthetic primer
ctgctagattcaagtcatcatgc

SEQ ID NO: 41 - DNA, 21 bp, Artificial Sequence
  Description of Artificial Sequence Synthetic primer
ctcgtgatggggctgatcttc

SEQ ID NO: 42 - DNA, 22 bp, Artificial Sequence
  Description of Artificial Sequence Synthetic primer
atctcacaccaatccgggaggt

SEQ ID NO: 43 - DNA, 540 bp, Sus sp.
  CDS (1)..(537)
atggggctgatcttcgctaaactgtggagcctcttctgtaaccaagagcacaaagtaattatagtgggactggataacgcagggaagaccactattctttatcagttcttaatgaatgaagtggttcatacatctccaactataggaagcaatgttgaagaaatagttgtgaagaacactcattttctcatgtgggatattggtggtcaagagtcactgcggtcatcctggaacacgtattattcaaacacagagttcatcattcttgtggttgatagcattgacagggaacgactagctattacgaaagaagaattatacagaatgttggctcatgaggatttacggaaggctgcagtccttatctttgccaataaacaggatatgaaagggtgcatgacagcagctgaaatctccaaatacctcaccctcagttcaattaaggatcatccgtggcatattcagtcctgctgtgctttaacaggagaagggttatgccaaggtctagagtggatgacctcccggattggtgtgagataa

SEQ ID NO: 44 - protein, 179 aa, Sus sp.
MGLIFAKLWSLFCNQEHKVIIVGLDNAGKTTILYQFLMNEVVHTSPTIGSNVEEIVVKNTHFLMWDIGGQESLRSSWNTYYSNTEFIILVVDSIDRERLAITKEELYRMLAHEDLRKAAVLIFANKQDMKGCMTAAEISKYLTLSSIKDHPWHIQSCCALTGEGLCQGLEWMTSRIGVR

SEQ ID NO: 45 - DNA, 22 bp, Artificial Sequence
  Description of Artificial Sequence Synthetic primer
aagcctgaagcttgatggataa

SEQ ID NO: 46 - DNA, 22 bp, Artificial Sequence
  Description of Artificial Sequence Synthetic primer
caattacagctttgctcctgtg

SEQ ID NO: 47 - DNA, 22 bp, Artificial Sequence
  Description of Artificial Sequence Synthetic primer
atagcattgacagggaacgact

SEQ ID NO: 48 - DNA, 22 bp, Artificial Sequence
  Description of Artificial Sequence Synthetic primer
gaactgagggtgaggtatttgg

SEQ ID NO: 49 - DNA, 332 bp, Artificial Sequence
  Description of Artificial Sequence Synthetic clone 5G9 from BMEC from swine brain
agcggagggcgcgcccatcagcctgctccgcagggtccggggcgctcttttcacctggaatattttgaaaacaattgccctgggtcasatgttgtccttgygtatatgtgggacagccatcaccagccagtatttggcagaaaaatacaaagtgaatacgcccatgcttcagagctttatcaactattgcttgctgtttctaatttatacaatgatgctggcatttcagtcaggtaataataaccttttatgcatcttgaaaaagaaatggtggaagtatatcctgctcggactggcagatgtggaagctaattacctgattgtcagagcgt

SEQ ID NO: 50 - DNA, 22 bp, Artificial Sequence
  Description of Artificial Sequence Synthetic primer
tgtatatgtgggacagccatca

SEQ ID NO: 51 - DNA, 22 bp, Artificial Sequence
  Description of Artificial Sequence Synthetic primer
gtccgagcaggatatacttcca

SEQ ID NO: 52 - DNA, 2319 bp, Sus sp.
  CDS (480)..(1466)
  modified_base (662): g or t
agtctctcttcagtccacacaagcctcagaagggtggcctacgggttggaatcgccccttcaatggcacctcagagacatctctgcatcgaaaggcaaaccgaacacgtccttaaggaggagacaccacagaaacatgtttccaggattctttaaggacgggaaagatagggaagaaaagaaacagaactataggaaataccttttacgatagtcaagagggagggagactaggtccaaggaggggtcagtcggtcctccccagttaacaaaggtcattgcttttcaggtggcataacctcgattcacctcaggtgctgattttagataaggaaccgtaagaacctgaaccgcctcttgggtgtctcctcaccccacgcagaagccccactgccaagacgaagaggaagagggcatttctcctccaactcctgctccggaggtgccaggaatattttgaaaacaattgccctgggtcagatgttgtccttgtgtatatgtgggacagccatcaccagccagtatttggcagaaaaatacaaagtgaatacgcccatgcttcagagctttatcaactattgcttgctgtttctaatttatacaatgatgctggcatttcagtcaggtaataataaccttttatgcatcttgaaaaagaaatgktggaagtatatcctgctcggactggcagatgtggaagctaattacctgattgtcagagcgtaccagtacacaactctaaccagtgtccagcttttggattgctttgggattcctgtgttgatggctctctcgtggtttattctttatgcaagatacagagtgatccacttcatcgctgtggctgtctgtctgttgggcgtaggaactatggttggtgcagacatattagcagggagagaagacaattcaggtagtgatgtgctgattggtgacgtcttggtccttcttggggcctccctctatgcagtttctaatgtgtgtgaagaatacatcgtgaagaagctgagccgacaggagtttttaggaatggtgggcttgtttggaacaattatcagtggcatacagctattgattgtggaatataaggatattgccagcattcactgggactggaaaattgccctactgtttgtagcatttgccctctgtatgttttgcctgtacagcttcatgccactggtgattaaagtcactagtgccacttctgtcaacctgggcatcctgacagctgacctctatagtcttttctttggactcttcctgtttggctataagttctcgggactctacatcctgtccttcgctgtcatcatggtggggttcattctgtactgttccacgccgacgcgcacggcagagccggctgaaagcagcgtgccaccaccagtcaccagcatcgggatcgacaacctgggcctgaagcttgaggagaacctcccggagacccactccgtggccttatagctggagaagaaggcacacacatgtactgcggctttctgggaagccgggagctatcacctgaataaagcagagcctgttgcctgctgaggggacacttggaaaatgatcagatgcagagtgaacactctggagcactggattggctctagtggttagattttatgaaggaatacaaatcaatgtatcaaaggtagaagtaccaaagtagagcagaagctaaggcaaggattgtgtttttgtgtgtttagggaccaatgtgtattaacgtcagggagacaaggtgtgaggcccacactggggtctcagaggcacaagatgggaaagcaggatggggtggatactcaggtgtgaggcagcctcaggacagggcctgaaagcaggctgtccaggtaggctggttggtcggggaggggaagagcatcccaggatggtttgggattaggtttgctcagttggaggcatctgagttctgtcctgctgaggcagtgattgtctcatgggctagacgaggtctggtgactgattgcgtacatcaggaagatggagggtgcagcactggagaaatcctgagatacaagtgtagaaccatagaagcagcacagcggatccttctcccaattgttactacactaatcttagcaaataatgtgccatgagatttttatgagacttcttcaaaacaaagttaacaggaagcatcattatgatatcaactaccaagcagtatgccmctttacacagatgctctatgtaaattttgggggggtaaaaatataataaaggaatcgagggtaaatgttcatattattaaaaatttttgatttcatagaaaaaaaaaaaaaaaaaaaaaaaa

SEQ ID NO: 53 - protein, 328 aa, Sus sp.
  MOD_RES (61): Cys or Trp
MLSLCICGTAITSQYLAEKYKVNTPMLQSFINYCLLFLIYTMMLAFQSGNNNLLCILKKKXWKYILLGLADVEANYLIVRAYQYTTLTSVQLLDCFGIPVLMALSWFILYARYRVIHFIAVAVCLLGVGTMVGADILAGREDNSGSDVLIGDVLVLLGASLYAVSNVCEEYIVKKLSRQEFLGMVGLFGTIISGIQLLIVEYKDIASIHWDWKIALLFVAFALCMFCLYSFMPLVIKVTSATSVNLGILTADLYSLFFGLFLFGYKFSGLYILSFAVIMVGFILYCSTPTRTAEPAESSVPPPVTSIGIDNLGLKLEENLPETHSVAL

SEQ ID NO: 54 - DNA, 407 bp, Artificial Sequence
  Description of Artificial Sequence Synthetic clone 5E7 from BMEC from swine brain
acagactgagatttagatgtttcattggccgtctgaagaggtgtggcttgtcttttatatagagatctacattataaaatactccgtgaagaaaaacacaccaaacgaaagagattttaagaatttggcacagttagtccctttgtgtaatctgaactcttctagctgctgaatatcttgaagtcattcctgttcactgaagtctttctgattgagctggttgaatactttgaaaaatgatgcgttctagctgttgaaatggatttcccaataggggttcctgcatattacctgtatagtagctctatgcatatgtttctgtgcatgctctctacccagttgtaaggtgtcactgtattttaactgttgcacttgtcaactttcaataaagcatataaaatgttggt

SEQ ID NO: 55 - DNA, 1905 bp, Artificial Sequence
  Description of Artificial Sequence cDNA of TSC-22 from BMEC from swine brain
agtctagagcctagtggagcccggctgccgacctgggagccttctccgcacagcagttggatctgcatcttcccggaatcgccaagccccagaagccgggtttctttcaattagggttgctgttttctgttcctccctgagccgcataaagctagaagatttttatctagctcaaacaaggcctctagaattccctcttttttaatttttttcctgcgagggtgttttttggctgcaattgcatgaaatcccaatggtgtagaccagtggcgatggatctaggagtttaccaactgagacatttttcaatttctttcttgtcatccttgctcgggactgaaaacgcctctgtgagacttgacaatagctcttctggtgcaagtgtggtagctattgacaacaaaatcgagcaagctatggatctggtgaaaagccatttgatgtatgcagttagagaggaagtggaggtcctcaaagagcaaatcaaagaactaatagagaaaaattcccagctggagcaggaaaacaatctgctgaagacactggccagtccggagcagcttgcccagttccaggcccagctgcagactggctccccgccggccaccacacagccccaggggaccacacagcccccggcccagccagcgtcccagggctcaggaccgaccgcgtagcctcctaggcccccccgcagaactggctgctgctgtctgaaccgactgaccgaccgaccgaccggagaggatgtgctgggggaggggggggtccgcctccaccacggtcacccatttcaatgctcagctgcgaaagagacgtgagactgacatatgccattatctcttttttccagtattaaaccctcatgtgcttttggcttgaagaagtttcttagttgggcgacttaaaggttaaccagagaattagcatggatgtactgggacctcatgcagcggggcagatccgtgagaaatggtttcattcatgctgaggagctgtgtgcctttccgcccctcccctgctccgcacccccacctccacccccacccctacccctacccccacctccgagaggtcgtcgtgcttgctcctggcgtgctgcgcgcagtccccaagccgtggagcgccactggactctcctctcgctcctcccccacgaggaaccggaaaggggggtgaaagtcaagaccgaagcttcatctcacctcggaggaggggaaacgtaggtcattgtacacgttgacgactgtcaccaaaatccataaaaaaacgaaacaaaaacccaagagtactgtgcctcttcccaaagcaagggatgacgcgggactattccagagtgactgaagggtgacaggtagctggcacctcggctatcaacgtgaaggyggttttgctcattgtatatttgtgtatgtaggtgtaactattttgtacaatagaggactgtaactactatttagcttgtacagactgagatttagatgtttcattggccgtctgaagargtgtggcttgtcttttatatagagatctacattataaaatactccgtgaagaaaaacacaccaaacgaaagagattttaagaatttggcacagttagtccctttgtgtaatctgaactcttctagctgctgaatatcttgaagtcasttcctgttcactgaagtctttctgattgagctggttgaatactttgaaaaatgatgcgttctagctgttgaaatggatttcccaataggggttcctgcatattacctgtatagtagctctatgcatatgtttctgtgcatgctctctacccagttgtaaggtgtcactgtattttaactgttgcacttgtcaactttcaataaagcatataaaatgttggtvmaaaaaaaaaaaaaaaaaaaaaaaaaaaaaa

SEQ ID NO: 56 - protein, 144 aa, Artificial Sequence
  Description of Artificial Sequence Amino acid sequence of TSC-22 from BMEC from swine brain
MKSQWCRPVAMDLGVYQLRHFSISFLSSLLGTENASVRLDNSSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGPTA

SEQ ID NO: 57 - DNA, 22 bp, Artificial Sequence
  Description of Artificial Sequence Synthetic primer
aagaggtgtggcttgtctttta

SEQ ID NO: 58 - DNA, 24 bp, Artificial Sequence
  Description of Artificial Sequence Synthetic primer
tttttcaaagtattcaaccagctc

SEQ ID NO: 59 - protein, 157 aa, Homo sapiens
MLVLLAGIFVVHIATVIMLFVSTIANVWLVSNTVDASVGLWKNCTNISCSDSLSYASEDALKTVQAFMILSIIFCVIALLVFVFQLFTMEKGNRFFLSGATTLVCWLCILVGVSIYTSHYANRDGTQYHHGYSYILGWICFCFSFIIGVLYLVLRKK

SEQ ID NO: 60 - protein, 160 aa, Mus sp.
MLVLLAGLFVVHIATAIMLFVSTIANVWMVADYANASVGLWKNCTGGNCDGSLSYGNEDAIKAVQAFMILSIIFSIISLVVFVFQLFTMEKGNRFFLSGSTMLVCWLCILVGVSIYTHHYAHSEGNFNSSSHQGYCFILTWICFCFSFIIGILYMVLRKK

SEQ ID NO: 61 - protein, 238 aa, Homo sapiens
MAARTGHTALRRVVSGCRPKSATAAGAQAPVRNGRYLASCGILMSRTLPLHTSILPKEICARTFFKITAPLINKRKEYSERRILGYSMQEMYDVVSGVEDYKHFVPWCKKSDVISKRSGYCKTRLEIGFPPVLERYTSVVTLVKPHLVKASCTDGRLFNHLETIWRFSPGLPGYPRTCTLDFSISFEFRSLLHSQLATLFFDEVVKQMVAAFERRACKLYGPETNIPRELMLHEVHHT

SEQ ID NO: 62 - protein, 242 aa, Mus sp.
MIMAARTSQRALARVASGCHPKSTTVTEAPARGSARDVRHLAACGVLINRTLPPCAAVLPKEICARTFFRISAPLVNKRKEYSERRILGYSMQEMYDVVSGMEDYQHFVPWCKKSDIISRRSGYCKTRLEVGFPPVLERYTSIVTLVKPHLVKASCTDGKLFNHLETIWRFSPGLPGYPRTCTLDFSISFEFRSLLHSQLATLFFDEVVKQMVAAFERRACKLYGPETNIPRELMLHEIHHT

SEQ ID NO: 63 - protein, 310 aa, Homo sapiens
MGNQVEKLTHLSYKEVPTADPTGVDRDDGPRIGVSYIFSNDDEDVEPQPPPQGPDGGGLPDGGDGPPPPQPQPYDPRLHEVECSVFYRDECIYQKSFAPGSAALSTYTPENLLNKCKPGDLVEFVSQAQYPHWAVYVGNFQVVHLHRLEVINSFLTDASQGRRGRVVNDLYRYKPLSSSAVVRNALAHVGAKERELSWRNSESFAAWCRYGKREFKIGGELRIGKQPYRLQIQLSAQRSHTLEFQSLEDLIMEKRRNDQIGRAAVLQELATHLHPAEPEEGDSNVARTTPPPGRPPAPSSEEEDGEAVAH

SEQ ID NO: 64 - protein, 292 aa, Homo sapiens
MGNQLDRITHLNYSELPTGDPSGIEKDELRVGVAYFFSDDEEDLDERGQPDKFGVKAPPGCTPCPESPSRHQHHLLHQLVLNETQFSAFRGQECIFSKVSGGPQGADLSVYAVTALPALCEPGDLLELLWLQPAPEPPAPAPHWAVYVGGGQIIHLHQGEIRQDSLYEAGAANVGRVVNSWYRYRPLVAELVVQNACGHLGLKSEEICWTNSESFAAWCRFGKREFKAGGEVPAGTQPPQQQYYLKVHLGENKVHTARFHSLEDLIREKRRIDASGRLRVLQELADLVDDKE

SEQ ID NO: 65 - protein, 307 aa, Homo sapiens [sequence omitted]

SEQ ID NO: 66 - protein, 309 aa, Mus sp.
MAALAPLPPLPAQFKSIQHHLRTAQEHDKRDPVVAYYCRLYAMQTGMKIDSKTPECRKFLSKLMDQLEALKKQLGDNEAVTQEIVGCAHLENYALKMFLYADNEDRAGRFHKNMIKSFYTASLLIDVITVFGELTDENVKHRKYARWKATYIHNCLKNGETPQAGPVGIEEENDVEENEDVGATSLPTQPPQPSSSSAYDPSNLAPGSYSGIQIPPGAHAPANTPAEVPHSTGVTSNAVQPSPQTVPAAPAVDPDLYTASQGDIRLTPEDFARAQKYCKYAGSALQYEDVGTAVQNLQKALRLLTTGRE

SEQ ID NO: 67 - protein, 450 aa, Homo sapiens
MAAPEPLRPRLCRLVRGEQGYGFHLHGEKGRRGQFIRRVEPGSPAEAAALAGDRLVEVNGVNVEGETHHQVVQRIKAVEGQTRLLVVDQETDEELRRRQLTCTEEMAQRGLPPAHDPWEPKPDWAHTGSHSSEAGKKDVSGPLRELRPRLCHLRKGPQGYGFNLHSDKSRPGQYIRSVDPGSPAARSGLRAQDRLIEVNGQNVEGLRHAEVVASIKAREDEARLLVVDPETDEHFKRLRVTPTEEHVEGPLPSPVTNGTSPAQLNGGSACSSRSDLPGSDKDTEDGSAWKQDPFQESGLHLSPTAAEARRRLEPCESTSARHRWTGTGSVKSSATSEPLPACLGTLGPLPHGPWASACPELPQPQWTGGWSCHCPEISPSPGEPPSCPCPPGTGGLWQQDRGRETQRCERESETETERERERHRERQRESERARGSRGARAFAALPGPAD

SEQ ID NO: 68 - protein, 327 aa, Homo sapiens
MLSLCICGTAITSQYLAERYKVNTPMLQSFINYCLLFLIYTVMLAFRSGSDNLLVILKRKWWKYILLGLADVEANYVIVRAYQYTTLTSVQLLDCFGIPVLMALSWFILHARYRVIHFIAVAVCLLGVGTMVGADILAGREDNSGSDVLIGDILVLLGASLYAISNVCEEYIVKKLSRQEFLGMVGLFGTIISGIQLLIVEYKDIASIHWDWKIALLFVAFALCMFCLYSFMPLVIKVTSATSVNLGILTADLYSLFVGLFLFGYKFSGLYILSFTVIMVGFILYCSTPTRTAEPAESSVPPVTSIGIDNLGLKLEENLQETHSAVL

SEQ ID NO: 69 - protein, 328 aa, Sus sp.
MLSLCICGTAITSQYLAEKYKVNTPMLQSFINYCLLFLIYTMMLAFQSGNNNLLCILKKKWWKYILLGLADVEANYLIVRAYQYTTLTSVQLLDCFGIPVLMALSWFILYARYRVIHFIAVAVCLLGVGTMVGADILAGREDNSGSDVLIGDVLVLLGASLYAVSNVCEEYIVKKLSRQEFLGMVGLFGTIISGIQLLIVEYKDIASIHWDWKIALLFVAFALCMFCLYSFMPLVIKVTSATSVNLGILTADLYSLFFGLFLFGYKFSGLYILSFAVIMVGFILYCSTPTRTAEPAESSVPPPVTSIGIDNLGLKLEENLPETHSVAL

SEQ ID NO: 70 - protein, 11 aa, Homo sapiens
DGSAWKQDPFQ